(19) United States
(12) Patent Application Publication
(10) Pub. No.: US 2019/0328681 A1
(43) Pub. Date: Oct. 31, 2019

(54) TRANSDERMAL PATCH

(71) Applicant: BUZZZ PHARMACEUTICALS LIMITED, Raheny Dublin (IE)

(72) Inventor: Kirti VALIA, East Windsor, NJ (US)

(21) Appl. No.: 16/070,574

(22) PCT Filed: Jan. 18, 2017
(86) PCT No.: PCT/EP2017/051013

§ 371 Date: Jul. 17, 2018

(30) Foreign Application Priority Data
Jan. 18, 2016 (GB) 1600920.1

(51) Int. Cl. A61K 9/70; A61K 47/12; A61K 31/485; A61K 47/38

(52) U.S. Cl. CPC A61K 9/7092; A61K 9/7061; A61K 47/38; A61K 31/485; A61K 47/12

(57) ABSTRACT

The present invention relates to an abuse deterrent transdermal patch, particularly transdermal patch for the transdermal administration of an opioid analgesic. The present invention also relates to processes for the preparation of the transdermal patches defined herein, as well as to the use of these patches for the treatment of pain.

### CROSS-REFERENCE TO RELATED APPLICATIONS

This application is a U.S. national phase of PCT/EP2017/051013, filed on Jan. 18, 2017, which claims priority to United Kingdom Patent Application No. 1600920.1, filed on Jan. 18, 2016, the disclosure of each of which is hereby incorporated by reference in its entirety.

### FIELD OF THE INVENTION

The present invention relates to a transdermal patch. More specifically, the present invention relates to an abuse deterrent transdermal patch for the transdermal administration of an opioid analgesic. The present invention also relates to processes for the preparation of the transdermal patches defined herein, as well as to the use of these patches for the treatment of pain.

### BACKGROUND OF THE INVENTION

Opioid analgesics are widely used in the clinic to treat moderate to severe pain. However, despite their clinical efficacy, opioid analgesics do suffer from some major drawbacks. One major drawback is that prolonged opioid analgesic use can lead to dependence, which gives rise to withdrawal symptoms if the opioid analgesic treatment is stopped abruptly. This opioid dependence can make opioid analgesics very addictive and prone to abuse. In addition, opioid analgesics are also well known for their ability to produce a feeling of euphoria, motivating some to use opioids recreationally.

The prevalence of opioid analgesic abuse is a major problem and the Food and Drug Administration (FDA) in the United States has initiated a program to encourage manufacturers of extended release and transdermal opioid formulations to consider innovative strategies to reduce the risk of abuse, and thereby encourage safe opioid use.

The transdermal delivery of opioid analgesics is a convenient and effective way to deliver opioid analgesics. However, there remains a need for improved approaches for the transdermal delivery of opioid analgesics. In addition, there is a need for novel abuse-deterrent transdermal patches which prevent, or substantially reduce, the risk of opioid abuse.

Aspects of the invention were devised with the foregoing in mind.

### SUMMARY OF THE INVENTION

In a first aspect, there is provided a transdermal patch comprising a multilaminate, wherein said multilaminate comprises:

(i) a first layer, comprising:
an opioid analgesic, or a pharmaceutically acceptable salt thereof;
a pharmaceutically acceptable pressure sensitive adhesive; and
optionally a penetration enhancer;
   (ii) a second layer, comprising:
an opioid antagonist, or a pharmaceutically acceptable salt thereof; and
a pharmaceutically acceptable adhesive;
   (iii) a barrier layer, comprising:
a water soluble cellulose derivative and a water insoluble cellulose derivative;
wherein said barrier layer is disposed between the first and second layers, and wherein the barrier layer substantially prevents the opioid antagonist diffusing from the second layer to the first layer during use;
with the proviso that the barrier layer is devoid of polyethylene glycol.

The transdermal patches of the present invention are abuse-deterrent patches. The barrier layer serves to prevent, or substantially prevent, the opioid antagonist present in the second layer from permeating through to the first layer during normal use of the patch. As a consequence, little or no opioid antagonist permeates into the skin of a patient during normal use. If the patch is misused, however, for example by an individual wishing to extract the opioid analgesic from the patch by immersing it in certain solvents, such as water, ethanol or acetone, or by chewing the patch, then the opioid antagonist present in the second layer will be released in an amount sufficient to antagonise some or all of the effects of the opioid analgesic that is extracted. This means that the extracted opioid analgesic will not exhibit the pharmacological effects expected.

In order to effectively antagonise the effects of the opioid analgesic released from the transdermal patch in certain solvents, it is necessary for the ratio of opioid analgesic to opioid antagonist to be within a certain range. The range required will depend on the particular antagonist used. Typically, the transdermal patches of the present invention release the opioid antagonist at a ratio of 60:1 to 1:60 (opioid analgesic:opioid antagonist) when the patch is immersed in water, acetone, ethanol or phosphate buffer (e.g. at a pH of 6-7) for a period of greater than 30 seconds, or greater than 1 minute, or greater than 2 minutes, typically for a period of greater than 30 minutes, more typically for a period of greater than 1 hour, and most typically for a period of greater than 2 hours.

Suitably, the transdermal patches of the present invention release the opioid antagonist at a ratio of 10:1 to 1:10 (opioid analgesic:opioid antagonist) when the patch is immersed in water, acetone, ethanol or phosphate buffer (e.g. at a pH of 6-7) for a period of greater than 30 seconds, or greater than 1 minute, or greater than 2 minutes, typically for a period of greater than 30 minutes, more typically for a period of greater than 1 hour, and most typically for a period of greater than 2 hours.

More suitably, the transdermal patches of the present invention release the opioid antagonist at a ratio of 4:1 to 1:4 (opioid analgesic:opioid antagonist) when the patch is immersed in water, acetone, ethanol or phosphate buffer (e.g. at a pH of 6-7) for a period of greater than 30 seconds, or greater than 1 minute, or greater than 2 minutes, typically for a period of greater than 30 minutes, more typically for a period of greater than 1 hour, and most typically for a period of greater than 2 hours.

In a second aspect, the present invention provides a transdermal patch as herein defined for use in therapy.

In another aspect, the present invention provides a transdermal patch as herein defined for use in the treatment of a condition selected form the group consisting of opioid dependence, alcohol dependence, polydrug addiction, pain, cocaine addiction, eating disorders (e.g., binge eating) and treatment-resistant depression.

In another aspect, the present invention provides a method of treating a condition selected form the group consisting of opioid dependence, alcohol dependence, polydrug addiction, pain, cocaine addiction, eating disorders (e.g., binge eating) and treatment-resistant depression in a subject in need of such treatment, said method comprising administering a transdermal patch as defined herein.

In a further aspect, the present invention provides a transdermal patch obtained, directly obtained, or obtainable by a process defined herein.

### DETAILED DESCRIPTION OF THE INVENTION

### Transdermal Patch of the First Aspect of the Invention

As indicated above, in a first aspect, the present invention provides a transdermal patch comprising a multilaminate, wherein said multilaminate comprises:

(i) a first layer, comprising:
an opioid analgesic, or a pharmaceutically acceptable salt thereof;
a pharmaceutically acceptable pressure sensitive adhesive; and
optionally a penetration enhancer;
   (ii) a second layer, comprising:
an opioid antagonist, or a pharmaceutically acceptable salt thereof; and
a pharmaceutically acceptable adhesive;
   (iii) a barrier layer, comprising:
a water soluble cellulose derivative and a water insoluble cellulose derivative;
wherein said barrier layer is disposed between the first and second layers, and wherein the barrier layer substantially prevents the opioid antagonist diffusing from the second layer to the first layer during use;
with the proviso that the barrier layer is devoid polyethylene glycol.

The first layer has a first surface that contacts the barrier layer and a second opposing surface that contacts the skin during use. The opioid analgesic present in the first layer diffuses or permeates into the skin over time to provide the desired analgesic effect.

Similarly, the second layer has a first surface which contacts the barrier layer and a second opposing surface.

The barrier layer prevents or substantially prevents the opioid antagonist present in the second layer from permeating through to the first layer during normal use of the patch. As a consequence, little or no opioid antagonist permeates into the skin of a patient during normal use. If the patch is misused, however, for example by an individual wishing to extract the opioid analgesic from the patch using certain solvents such as water, ethanol or acetone, or by chewing the patch, then the opioid antagonist present in the second layer will be released to antagonise the effects of the opioid analgesic, thereby preventing the subsequent misuse of the extracted opioid analgesic.

The transdermal patch suitably further comprises a barrier layer that extends over the second layer (i.e. over the second surface of the second layer). The second surface of the first layer is suitably covered with a peelable release liner that extends across the entire second surface of the first layer, but which can be removed to expose the second surface of the first layer prior to application of the patch to the skin.

### Backing Films

Suitably the backing film is occlusive. The backing film may be of any thickness, but is suitably between about 0.1 to 100 mil (2.5 μm to 2.5 mm) thick. Suitable materials include, but are not limited to, synthetic polymers including, for example, polyesters, polycarbonates, polyimides, polyethylene, poly(ethylene teraphthalate), polypropylene, polyurethanes and polyvinylchlorides. The barrier layer may also be a laminate comprising additional layers that may include vapour deposited metal, such as aluminium, additional synthetic polymers, and other materials, to enable a heat seal, such as EVA copolymer. Suitably, the backing film comprises occlusive Scotchpak 9730®, Scotchpak 9732° or Scotchpak 9733° obtainable from 3M. Most suitably, the barrier layer comprises Scotchpak 9733®.

In one embodiment, the backing film has a thickness of 0.1 to 50 mil (2.5 μm to 1.25 mm), more suitably, 1-20 mil (25 μm to 0.635 mm), even more suitably 1-10 mil (25 μm to 0.25 mm), and most suitably 1-5 mil (25 μm to 127 μm).

### Release Liner

The release liner is typically disposed on an opposite surface of the multi-laminate (i.e. the second surface of the first layer) to the barrier layer and provides a removable protective or impermeable layer, usually but not necessarily rendered non-stick so as to not adhere to the first layer. The release liner serves to protect the first layer during storage and transit, and is intended to be removed prior to application to the skin. The release liner may be formed from the same materials used for the barrier layer, but may be formed from metal foils, Mylar®, polyethylene terephthalate, siliconized polyester, fumed silica in silicone rubber, polytetrafluoroethylene, cellophane, siliconized paper, aluminized paper, polyvinyl chloride film, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene.

Suitably, the release liner is an occlusive or semi-occlusive backing film being compatible with the pharmaceutically-acceptable adhesive present in the pharmaceutical formulation layer.

Suitably, the release liner may be selected from Scotchpak 9741®, Scotchpak 1022®, Scotchpak 9742®, Scotchpak 9744®, Scotchpak 9748° and Scotchpak 9755®, all of which are obtainable from 3M and comprise fluoropolymers coated onto polypropylene or polyester film. Other suitable release liners made by other manufacturers may also be used. The release liner may be of any thickness known in the art. Suitably the release liner has a thickness of about 0.01 mm to about 2 mm.

In one embodiment, the release liner is selected from Scotchpak 9741®, Scotchpak 1022° or Scotchpak 9744®. Suitably, the release liner is Scotchpak 9744®.

In another embodiment, the release liner has a thickness of 0.1 to 50 mil (2.5 μm to 1.25 mm), more suitably, 1-20 mil (25 μm to 0.635 mm), even more suitably 1-10 mil (25 μm to 0.25 mm), and most suitably 1-5 mil (2.5 μm to 127 μm).

The container or closure system may be made from a range of materials suitable for protecting the packaged transdermal patch from moisture and light.

### The First Layer

In one embodiment, the first layer has a dry thickness of 0.1-100 mil (2.5 μm to 2.5 mm), suitably, 1-50 mil (25 μm to 1.25 mm), more suitably 2-20 mil (50 μm to 0.635 mm), yet more suitably 5-20 mil (0.125 mm to 0.635 mm), even most suitably 10-15 mil (0.25 mm to 0.38 mm) and most suitably, 10-12 mil (0.25 mm to 0.305 mm).

In an embodiment, the oxymorphone layer has a dry thickness of 1 to 15 mil, 1 to 12 mil, 10 to 12 mil, 2 to 3 mil or 2.5 to 3 mil.

### Opioid Analgesic

The first layer of the transdermal patches of the present invention comprises an opioid analgesic. Any suitable opioid analgesic may be used.

The opioid analgesic can be selected from the group comprising alfentanil, allylprodine, alphaprodine, anileridine, benzylmorphine, bezitramide, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydroxypethidine, isomethadone, ketobemidone, levorphanol, levomethadyl, levophenacylmorphan, lofentanil, meperidine, metazocine, methadone, metopon, morphine, myrophine, narceine, nicomorphine, norlevorphanol, normethadone, normorphine, norpipanone, opium, oxycodone, oxymorphone, papaveretum, phenadoxone, phenomorphan, phenazocine, phenoperidine, piminodine, piritramide, propheptazine, promedol, properidine, propoxyphene, sufentanil, tilidine, tramadol, buprenorphine, butorphanol, dezocine, meptazinol, nalbuphine, nalorphine, pentazocine, tapentadol and salts of the foregoing. In another embodiment, the composition comprises pharmaceutically acceptable prodrugs of the opioid analgesic.

In an embodiment, the opioid analgesic is selected from morphine, codeine, thebaine, diacetylmorphine (morphine diacetate; heroin), nicomorphine (morphine dinicotinate), dipropanoylmorphine (morphine dipropionate), desomorphine, acetylpropionylmorphine, dibenzoylmorphine, diacetyldihydromorphine, hydromorphone, hydrocodone, oxycodone, oxymorphone, ethylmorphine and buprenorphine, fentanyl, pethidine, levorphanol, methadone, tramadol, tapentadol and dextropropoxyphene.

In a further embodiment, the opioid analgesic is selected from buprenorphine, fentanyl, hydromorphone, oxycodone, tapentadol or oxymorphone, or a pharmaceutically acceptable salt thereof.

Any suitable opioid containing transdermal formulation may be used to form the first layer in the compositions of the present invention. Examples of suitable first layers for inclusion in the transdermal patches of the present invention include the fentanyl containing transdermal formulations described in EP 1381352 B1, U.S. Pat. Nos. 6,139,866, 5,985,317, 5,762,952 and 5,474,783, and tapentadol-containing transdermal formulations described in WO 2014012653, the entire contents of which are incorporated herein by reference.

In an embodiment, the opioid analgesic is oxymorphone, fentanyl, or a pharmaceutically acceptable salt thereof.

In a particular embodiment, the opioid analgesic is oxymorphone, or a pharmaceutically acceptable salt thereof. Suitably, the opioid analgesic is a non-salt (i.e. free base) form of oxymorphone.

The amount of opioid analgesic present in the first layer of the patches of the present invention will depend on how soluble it is in the pharmaceutically-acceptable adhesive and excipients present in this layer and how much of the opioid analgesic is required in order to achieve the desired therapeutic effect. Typically, the opioid analgesic will be present at an amount of 1-15% w/w in the first layer.

In one embodiment, the amount of opioid analgesic present is 1-10% w/w in the first layer. Suitably, the amount of opioid analgesic present is 2-5% w/w, more suitably 2-4% w/w, and most suitably 2-3% w/w in the first layer. In an embodiment, the amount of opioid analgesic present is 2.0-3.0% w/w in the first layer. In a further embodiment, the amount of opioid analgesic present is 2.2-2.8% w/w in the first layer. In yet another embodiment, the amount of opioid analgesic present is 2-2.6% w/w in the first layer. For example, the first layer of the transdermal patch may comprise 2.0%, 2.5% or 3% w/w of opioid analgesic.

The opioid analgesic may be present in the form of a pharmaceutically acceptable salt thereof. A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Although the opioid analgesic may be present in the form of a salt, a person skilled in the art will appreciate that the opioid analgesic needs to be in a form that has a suitable lipophilic/hydrophilic balance so as to enable good permeation through the skin. In some embodiments of the invention, the opioid analgesic is present in a non-salt form, i.e. as a free base or acid

In an embodiment, the opioid analgesic is present in a non-salt form, i.e. as a free base.

In another embodiment, the ratio of opioid analgesic:opioid antagonist in the transdermal patch is between 10:1 and 1:10. Suitably, the ratio of opioid analgesic:opioid antagonist is between 10:1 and 1:6, more suitably, the ratio is between 4:1 and 1:4, yet more suitably, the ratio is between 2:1 and 1:2, even more suitably, the ratio is between 1:1 and 1:2, most suitably, the ratio is between 1:1 and 1:1.5.

### Pharmaceutically Acceptable Pressure Sensitive Adhesive

The pharmaceutically acceptable pressure sensitive adhesive present in the first layer is selected both in terms of its ability to solubilise the opioid analgesic, and its adhesive tack and peel properties.

In one embodiment, the adhesive has an opioid analgesic solubility in excess of 2.0% w/w at room temperature.

In another embodiment, the pharmaceutically acceptable pressure sensitive adhesive has an opioid analgesic solubility of up to 5% w/w at room temperature. Typically, the pharmaceutically acceptable pressure sensitive adhesive has an opioid analgesic solubility of up to 4% w/w at room temperature, more typically, up to 3.5% w/w or 3.0% w/w.

Typically, the total amount of adhesive will constitute between 58 and 99% w/w of the first layer. Suitably, total amount of adhesive will constitute between 65 and 99% w/w of the first layer, more suitably between 72 and 97% w/w, yet more suitably between 87 and 95% w/w and most suitably between 92 and 95% w/w.

Any suitable adhesive material or combination of adhesive materials may be used. Such materials are suitably pressure sensitive adhesives.

Examples of suitable pressure sensitive adhesives include polymer and copolymers of polyacrylates, polysiloxanes, polyisobutylene, polyisoprene, polybutadiene, ethylene-vinyl acetate and styrenic block polymers, such as styrene-isoprene-styrene block copolymer, styrene-butadiene-styrene copolymer, styrene-ethylenebutene-styrene copolymers, styrene-ethylene/propylene-styrene copolymers and di-block analogs thereof. Examples of polyacrylates include, but are not limited to, acrylic acids, alkyl acrylates and methacrylates; for example, acrylic acid, methacrylic acid, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate, methoxyethyl methacrylate, vinylacetate/ethylene acrylate and the like. Additional examples of appropriate acrylic adhesives suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, New York (1989).

Other useful pressure sensitive adhesives (PSA) can include mixtures of different polymers or mixtures of polymers such as synthetic rubber polyisobutylene (PIB), The PIB adhesives normally include a tackifier such as polybutene oil and resins such as the ESCOREZ® resins available from Exxon Chemical. Other useful rubber-based pressure-sensitive adhesives include hydrocarbon polymers such as natural and synthetic polyisoprene, polybutylene and polyisobutylene, styrene/butadiene polymers styrene-isoprene-styrene block copolymers, hydrocarbon polymers such as butyl rubber, halogen-containing polymers such as polyacrylic-nitrile, polytetrafluoroethylene, polyvinylchloride, polyvinylidene chloride, and polychlorodiene, and other copolymers thereof. Polyisobutylene polymers are available commercially under the trademark name VISTANEX® from Exxon Chemical.

Silicone-based pressure sensitive adhesives are also suitable for use in additional embodiments described herein. Suitable silicone-based pressure-sensitive adhesives can include those described in Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989), incorporated by reference in its entirety. Other useful silicone-based pressure sensitive adhesives are described in the following U.S. Pat. Nos. 4,591,622; 4,584,355; 4,585,836; and 4,655,767 which are hereby incorporated by reference in their entirety. Suitable silicone-based pressure-sensitive adhesives are commercially available and include the silicone adhesives sold under the trademarks BIO-PSA 7-4503, BIO-PSA 7-4603, BIO-PSA 7-4301, 7-4202, 7-4102, 7-4106, and BIO-PSA 7-4303 by Dow Corning Corporation, Medical Products, Midland, Mich. The commercially available silicones are sold under the trademark of BIO-PSA such as Bio-PSA 7-4102, 7-4202, 7-4302, 7-4101, 7-4201, 7-4301, 7-4303, 7-4503, 7-4603 by Dow Corning Cooperation. In one embodiment, amine-compatible Bio-PSA silicone adhesives are preferred. In a further embodiment, the preferred amine-compatible Bio-PSA silicone adhesive 7-4202 was employed in combination with acrylic adhesive such as Duro-tak 87-9301 manufactured by National Starch and Chemical Company.

In one embodiment a pressure sensitive adhesive is optionally used to assist in affixing a patch containing an opioid to be transdermally delivered to the subject. In a further embodiment, the pressure sensitive adhesive is present in a total amount by weight between about 58% and about 99%; between about 60% and about 95% and between about 70% and about 90% of the first layer. In a further embodiment the pressure sensitive adhesive layer is a mixture of two or more pressure sensitive adhesives.

In an embodiment, the adhesive is selected from acrylate/polyacrylate materials, rubbers and silicones.

In a further embodiment, the adhesive is selected from acrylate/polyacrylate materials and silicones.

In yet a further embodiment, the adhesive is mixture of an acrylate/polyacrylate material and a silicone material.

Suitably, the silicone adhesive comprises a composition of a silicone adhesive in a suitable solvent, for example ethyl acetate and/or hexane. As indicated above, examples of such adhesives includes those that are commercial available from Dow Corning® under the BIO-PSA® product range. These adhesives are compatible with amine containing drugs and are formed by a condensation reaction of silanol end-blocked polydimethylsiloxane (PDMS) with a silicate resin, and the residual silanol functionality is then capped with trimethylsiloxy groups to yield the chemically stable amine-compatible adhesives.

Particular examples of suitable silicone adhesives include BIO-PSA® 7-4502, 7-4302 and 7-4202 or mixtures thereof.

Suitably the silicone based adhesive represents 60-98% w/w of the first layer. More suitably, the silicone based adhesive represents 60-90% w/w of the first layer. Even more suitably, the silicone based adhesive represents 60-80% w/w of the first layer. Most suitably, the silicone based adhesive represents 70-80% w/w of the first layer.

In a particular embodiment, the pressure sensitive adhesive is a pressure sensitive polyacrylate adhesive.

In another embodiment, the pressure sensitive polyacrylate adhesive comprises an acrylate copolymer and/or an acrylate-vinyl acetate, such as Durotak-87-4098, Durotak-87-4287®, Durotak-87-2510®, Durotak-87-2287®, Duro-Tak 87-2677®, Durotak-87-2516®, Durotak-87-2525®, Duro-Tak 87-900A®, Duro-Tak 87-2074®, Duro-Tak 87-2054®, Duro-Tak 87-2052®, Duro-Tak 87-2196°, Duro-Tak 9301®, Duro-Tak 2054®, Duro-Tak 606A®, and/or Duro-Tak 202A®, obtainable from Henkel. Suitably, the polyacrylate adhesive comprises both an acrylate copolymer and an acrylate-vinyl acetate.

It will be appreciated that the pressure sensitive polyacrylate adhesive of the first layer may comprise a crosslinker.

In an embodiment the pressure sensitive polyacrylate adhesive has a viscosity of between 1600 and 19000 mPa. Suitably, the polyacrylate adhesive has a viscosity of between 4000 and 18000 mPa. More suitably, the polyacrylate adhesive has a viscosity of between 7000 and 18000 mPa. Most suitably, the polyacrylate adhesive has a viscosity of between 7500 and 8500 mPa.

Suitably, the pressure sensitive polyacrylate adhesive is obtained from Henkel.

Any suitable adhesive material or combination of adhesive materials as defined herein may be used.

In an embodiment, the pressure sensitive polyacrylate comprises no functional groups or a plurality of hydroxyl functional groups. Suitably, the pressure sensitive polyacrylate adhesive comprises a plurality of hydroxyl functional groups.

In another embodiment, the pressure sensitive polyacrylate adhesive comprises a plurality of hydroxyl functional groups and an acrylate-vinyl acetate.

In an embodiment, the pressure sensitive polyacrylate adhesive comprises a plurality of hydroxyl functional groups and has a viscosity of between 1600 and 19000 mPa. Examples of pressure sensitive polyacrylate adhesives comprising such properties include, but are not limited to, Durotak-87-4287®, Durotak-87-202A®, Durotak-87-2510®, Durotak-87-2287®, Durotak-87-2516° or Durotak-87-2525®, obtainable from Henkel.

In another embodiment, the pressure sensitive polyacrylate adhesive comprises a plurality of hydroxyl functional groups and an acrylate-vinyl acetate, and has a viscosity of between 4000 and 18000 mPa. Examples of pressure sensitive polyacrylate adhesives comprising such properties include, but are not limited to, Durotak-87-4287®, Durotak-87-2287®, Durotak-87-2516° or Durotak-87-2525®.

In another embodiment, the pressure sensitive polyacrylate adhesive comprises a plurality of hydroxyl functional groups and an acrylate-vinyl acetate, has a viscosity of between 7000 and 18000 mPa and is devoid of any crosslinker. Examples of pressure sensitive polyacrylate adhesives comprising such properties include, but are not limited to, Duro-Tak 87-2287® or Duro-Tak 87-4287®.

In a further embodiment, the pressure sensitive polyacrylate adhesive comprises a plurality of hydroxyl functional groups and an acrylate-vinyl acetate, has a viscosity of between 7500 and 8500 mPa and is devoid of any crosslinker. Examples of pressure sensitive polyacrylate adhesives comprising such properties include, but are not limited to Duro-Tak 87-4287®.

Suitably, the pressure sensitive polyacrylate adhesive is selected from Durotak-87-4287®, Durotak-87-2287®, Durotak-87-2516® or Durotak-87-2525®, more suitably, Duro-Tak 87-2287® or Duro-Tak 87-4287®, most suitably, Duro-Tak 87-4287®.

In one embodiment, a suitable volatile solvent is added to the adhesive to reduce viscosity and aid solvation. Suitable solvents may include, but are not limited to, isopropyl alcohol, methanol, ethanol and ethyl acetate.

### Penetration Enhancer

In an embodiment, the first layer further comprises a penetration enhancer.

The composition may comprise one or more penetration enhancers for transdermal drug delivery. Examples of penetration enhancers include C8-C22 fatty acids such as isostearic acid, octanoic acid, myristic acid and oleic acid; C8-C22 fatty alcohols such as oleyl alcohol and lauryl alcohol; lower alkyl esters of C8-C22 fatty acids such as ethyl oleate, isopropyl myristate (IPM), butyl stearate, and methyl laurate; di(lower)alkyl esters of C6-C22 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxy)ethanol (transcutol); diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes

In an embodiment, the penetration enhancer is selected from oleic acid, oleyl alcohol, myristic acid, lauryl alcohol, lauryl acetate, lauryl lactate, ethyl acetate, dimethyl isosorbide, isostearic acid or linoleic acid.

In a particular embodiment, the penetration enhancer is oleic acid or linoleic acid. Suitably, the penetration enhancer is linoleic acid.

In an embodiment, the first layer comprises one penetration enhancer. In another embodiment, the composition comprises two or more penetration enhancers.

The penetration enhancer is present in an amount sufficient to provide the desired physical properties and skin penetration profile for the composition.

For example, one or more pharmaceutically acceptable penetration enhancers can be present in a total amount by weight of 0.1-15% w/w of the first layer. In an embodiment, one or more pharmaceutically acceptable penetration enhancers are present in a total amount by weight between 2% and 12% w/w of the first layer, or between 4% and 10% w/w, or between 4% and 7% w/w, or between 4% and 6% w/w, or between 4.5% and 5.5 w/w, or between 4% and 5% w/w, or about 5% w/w.

### Hydrophilic Materials

In certain embodiments, the use of hydrophilic materials in the first layer may aid the skin absorption of the opioid analgesic or the solubility of the opioid analgesic in the adhesive. Suitably, the hydrophilic material, and the quantities in which it is added, should be non-toxic, non-irritating, non-allergenic, and compatible with the opioid analgesic and the other excipients herein described.

In one embodiment, the hydrophilic material will have a hydrophilic-lipophilic balance (HLB) of greater than 7. Examples of hydrophilic materials suitable for inclusion into the pharmaceutical formulation of the present invention include, but are not limited to, propylene glycol, dipropylene glycol, glycerol, polyethylene glycol, short chain water soluble esters of citric acid, acetic acid, hexylene glycol and alcohols, including diols and polyols.

The amount of hydrophilic material present is 0-20% w/w.

Suitably, when used, the hydrophilic material is present in the first layer in an amount of between 1.0% w/w and 20% w/w.

Suitably, the hydrophilic material, when present, is in an amount of between 0.5 and 10% w/w, and more suitably between 1 and 8% w/w.

Suitably, the hydrophilic material is propylene glycol or dipropylene glycol.

In an embodiment, the hydrophilic material is included in the first layer as part of a mixture including the opioid analgesic, the pharmaceutically acceptable adhesive and a penetration enhancer.

### Particular Embodiments of the First Layer

The following represent particular embodiments of the first layer:

| 1.1 | 1-15% | w/w | opioid analgesic (e.g. oxymorphone or fentanyl); |
|---|---|---|---|
| | 50-98% | w/w | pharmaceutically acceptable pressure sensitive adhesive; |
| | 0.1-15% | w/w | penetration enhancer; |
| | 0-20% | w/w | hydrophilic material (e.g. propylene glycol). |
| 1.2 | 1-15% | w/w | opioid analgesic (e.g. oxymorphone or fentanyl); |
| | 40-80% | w/w | Acrylate/polyacrylate adhesive (e.g. BIO-PSA 7-4502); |
| | 0-15% | w/w | Silicone adhesive (e.g. Duro-Tak 9301 ®) |
| | 2-12% | w/w | penetration enhancer; |
| | 0-10% | w/w | hydrophilic material (e.g. propylene glycol). |
| 1.3 | 1-6% | w/w | opioid analgesic (e.g. oxymorphone or fentanyl); |
| | 70-80% | w/w | Acrylate/polyacrylate adhesive (e.g. BIO-PSA 7-4502); |
| | 0-15% | w/w | Silicone adhesive (e.g. Duro-Tak 9301 ®) |
| | 5-12% | w/w | penetration enhancer; |
| | 0-8% | w/w | hydrophilic material (e.g. propylene glycol). |
| 1.4 | 1-10% | w/w | opioid analgesic (e.g. oxymorphone or fentanyl); |
| | 65-98% | w/w | pharmaceutically acceptable pressure sensitive polyacrylate |
| | | | adhesive (e.g. Duro-Tak 9301 ®, Duro-Tak 2054 ®, Durotak-87- |
| | | | 4287 ®, Durotak-87-2287 ®, Durotak-87-2516 ® or Durotak-87- |
| | | | 2525 ®); |
| | 0.1-15% | w/w | penetration enhancer selected from oleic acid or linoleic acid; |
| | 0-20% | w/w | hydrophilic material (e.g. propylene glycol). |
| 1.5 | 1-6% | w/w | opioid analgesic (e.g. oxymorphone or fentanyl); |
| | 72-97% | w/w | pharmaceutically acceptable pressure sensitive polyacrylate |
| | | | adhesive (e.g. Duro-Tak 9301 ®, Duro-Tak 2054 ®, Durotak-87- |
| | | | 4287 ®, Durotak-87-2287 ®, Durotak-87-2516 ® or Durotak-87- |
| | | | 2525 ®); |
| | 2-12% | w/w | penetration enhancer selected from oleic acid or linoleic acid; |
| | 0-10% | w/w | hydrophilic material (e.g. propylene glycol). |
| 1.6 | 1-6% | w/w | opioid analgesic (e.g. oxymorphone or fentanyl); |
| | 87-95% | w/w | pharmaceutically acceptable pressure sensitive polyacrylate |
| | | | adhesive comprising no functional groups or a plurality of hydroxyl |
| | | | functional groups (e.g. Durotak-87-4098 ®, Durotak-87-4287 ®, |
| | | | Durotak-87-2287 ®, Durotak-87-2516 ® or Durotak-87-2510 ®); |
| | 4-7% | w/w | penetration enhancer selected from oleic acid or linoleic acid. |
| 1.7 | 2-4% | w/w | opioid analgesic (e.g. oxymorphone or fentanyl); |
| | (e.g. 3% w/w) | | |
| | 91-95% | w/w | pharmaceutically acceptable pressure sensitive polyacrylate |
| | | | adhesive comprising a plurality of hydroxyl functional groups (e.g. |
| | | | Durotak-87-4287 ®, Durotak-87-2516 ® or Durotak-87-2510 ®); |
| | 4-5% | w/w | penetration enhancer selected from oleic acid or linoleic acid. |
| 1.8 | 2-3% | w/w | opioid analgesic (e.g. oxymorphone or fentanyl); |
| | 92-95% | w/w | pharmaceutically acceptable pressure sensitive polyacrylate |
| | | | adhesive comprising a plurality of hydroxyl functional groups, an |
| | | | acrylate-vinyl acetate and a viscosity of between 7000 and 18000 mPa |
| | | | (e.g. Durotak-87-4287); |
| | 4-5% | w/w | penetration enhancer selected from oleic acid or linoleic acid. |

Particular examples of the first layer are provided in the example defined hereinbelow.

### The Second Layer

The dry thickness of the second may be 0.1-100 mil (2.5 μm to 2.5 mm), suitably 1-50 mil (0.25 μm to 1.27 mm), more suitably 1-20 mil (0.25 μm to 0.51 mm), yet more suitably 1-15 mil (0.25 μm to 0.381 mm), even more suitably 5-15 mil (0.127 μm to 0.381 mm), and most suitably 10-15 mil (0.25 μm to 0.381 mm).

In an embodiment, the second layer has a dry thickness of 1 to 15 mil, 1 to 13 mil, 10 to 13 mil, 1 to 3 mil or 1 to 2 mil.

### Opioid Antagonist

Suitably, the opioid antagonist is present in an amount of 0.1-10% w/w, more suitably 0.1-5% w/w, yet more suitably 0.1-3% w/w, even more suitably 0.5-3% w/w and most suitably 1-3% w/w in the second layer.

The opioid antagonist is suitably selected from the group consisting of: naltrexone (“NTX”), 6-beta-naltrexol, nalbuphine, nalmefene, naloxone (“NLX”), cyclazocine, levallorphan, cyclorphan, oxilorphan and pharmaceutically acceptable salts and prodrugs thereof.

Suitably, the opioid antagonist is suitably selected from the group consisting of: naltrexone (“NTX”), naloxone (“NLX”) or nalmefene and pharmaceutically acceptable salts and prodrugs thereof.

Suitably, the opioid antagonist is naltrexone (“NIX”) or naloxone (“NLX”), or a pharmaceutically acceptable salt thereof. Most suitably, the opioid antagonist is naltrexone (“NIX”), or a pharmaceutically acceptable salt thereof.

In an embodiment, a proportion of the opioid antagonist is present in non-salt (e.g. free base) form and the remainder is present in the form of a pharmaceutically acceptable salt.

The amount of the non-salt (i.e. free base) form of the opioid antagonist present may be 5-100% w/w, more suitably 15 to 100% w/w, even more suitably 30-100% w/w and most suitably 55-100% w/w of the total opioid antagonist present in the second layer (and the balance is the salt (e.g. HCl salt) form).

In an embodiment, the opioid antagonist is present in non-salt (e.g. free-base) form.

The use of just the non-salt form of the antagonist, e.g. the free base of naltrexone or naloxone, has, in some instances, been found to result in some migration of the antagonist into first layer during use with certain barrier layers. Conversely, the use of just the salt form can prevent this migration during use, but then does not give the desired release rate of antagonist with the opioid when the patch is exposed to solvents such as water, phosphate buffered saline, ethanol and acetone.

The use of both the salt and non-salt forms together enables a balance between the migration through to the first layer during use and the achieving the required release ratio of antagonist to opioid analgesic when the patch is subject to abuse. This enables a larger number of barrier layers to be used.

The amount of opioid antagonist present in the second layer of the patches of the present invention will depend on how soluble it is in the pharmaceutically-acceptable adhesive and any other excipients present in this layer, and how much of the opioid antagonist is required relative to the opioid agonist in order to achieve the desired abuse deterrent effect. Typically, the opioid antagonist will be present at an amount of 0.1-5% w/w in the second layer.

In one embodiment, the opioid antagonist is present in an amount of 0.5-3% w/w or 1-3% w/w in the second layer.

### Pharmaceutically Acceptable Adhesive

The pharmaceutically-acceptable adhesive present in the second layer is selected both in terms of its ability to solubilise the opioid antagonist and its adhesive tack properties.

In one embodiment, the adhesive has an opioid antagonist solubility in excess of 2.5% w/w at room temperature.

Typically, the amount of adhesive is between 58 and 99% w/w of the second layer, suitably 70-99% w/w, more suitably 85-99% w/w, even more suitably 95-99% w/w, and most suitably 97-99% w/w.

Any suitable adhesive material or combination of adhesive materials as defined above in relation to the first layer may be used.

In an embodiment, an acrylate or polyacrylate adhesive material and/or a silicone adhesive as defined in relation to the first layer represents 50-98% w/w of the second layer, and more suitably 55-75% w/w, and even more suitably 60-70% w/w of the second layer.

Additional adhesives, such as, for example, polyvinylpyrollidinone (e.g. polyvinylpyrollidone K30) may also be present, for example in amount of between 0.5 to 15% w/w of the second layer, or 1 to 6% w/w, or 2 to 5% w/w.

In a particular embodiment, the pharmaceutically-acceptable adhesive is a pharmaceutically-acceptable polyacrylate adhesive.

In an embodiment, the pharmaceutically-acceptable polyacrylate adhesive comprises a plurality of carboxyl functional groups.

In another embodiment, the pharmaceutically-acceptable polyacrylate adhesive comprises an acrylate copolymer and/or an acrylate-vinyl acetate. Suitably, the polyacrylate adhesive comprises both an acrylate copolymer and an acrylate-vinyl acetate.

It will be appreciated that the pharmaceutically-acceptable polyacrylate adhesive of the first layer may comprise a crosslinker.

In an embodiment the pressure sensitive polyacrylate adhesive has a viscosity of between 1600 and 10000 mPa. Suitably, the polyacrylate adhesive has a viscosity of between 1600 and 7000 mPa. More suitably, the polyacrylate adhesive has a viscosity of between 1600 and 4000 mPa. Most suitably, the polyacrylate adhesive has a viscosity of between 2500 and 3500 m Pa.

Suitably, the pharmaceutically-acceptable polyacrylate adhesive is obtained from Henkel.

In an embodiment, the pharmaceutically-acceptable polyacrylate adhesive comprises a plurality of carboxy functional groups and has a viscosity of between 1600 and 10000 mPa. Examples of pharmaceutically-acceptable polyacrylate adhesives comprising such properties include, but are not limited to, Durotak-87-2054®, Durotak-87-235A®, Durotak-87-2353®, Durotak-87-2852®, Durotak-87-2051®, Durotak-87-2052®, Durotak-87-2677®, Durotak-87-2194®, Durotak-87-2196° or Durotak-87-2825®, obtainable from Henkel.

In another embodiment, the pharmaceutically-acceptable polyacrylate adhesive comprises a plurality of carboxy functional groups, an acrylate-vinyl acetate and has a viscosity of between 1600 and 7000 mPa. Examples of pharmaceutically-acceptable polyacrylate adhesives comprising such properties include, but are not limited to, Durotak-87-2054®, Durotak-87-2051®, Durotak-87-2052®, Durotak-87-2677®, Durotak-87-2194®, Durotak-87-2196® or Durotak-87-2825®.

In another embodiment, the pharmaceutically-acceptable polyacrylate adhesive comprises a plurality of carboxy functional groups, an acrylate-vinyl acetate and a crosslinker, and has a viscosity of between 2500 and 3500 mPa. Examples of pharmaceutically-acceptable polyacrylate adhesives comprising such properties include, but are not limited to, Duro-Tak 87-2054® or Duro-Tak 87-2194®.

Suitably, the pharmaceutically-acceptable polyacrylate adhesive is selected from Durotak-87-2054®, Durotak-87-2051®, Durotak-87-2052®, Durotak-87-2677®, Durotak-87-2194®, Durotak-87-2196® or Durotak-87-2825®, more suitably, Duro-Tak 87-2054® or Duro-Tak 87-2194®. Most suitably, Duro-Tak 87-2054®.

In one embodiment, a suitable volatile solvent is added to the adhesive to reduce viscosity and aid solvation. Suitable solvents may include, but are not limited to, isopropyl alcohol, methanol, ethanol and ethyl acetate.

### Hydrophilic Materials

The second layer may optionally comprise a hydrophilic material as defined hereinbefore in relation to the first layer.

The amount of hydrophilic material present in the second layer is 0-20% w/w.

Suitably, when used, the hydrophilic material is present in the second layer in an amount of between 1.0% w/w and 10% w/w.

Suitably, the hydrophilic material, when present, is in an amount of between 0.5 and 10% w/w, and more suitably between 1 and 8% w/w.

Suitably, the hydrophilic material is propylene glycol or dipropylene glycol.

### Cellulose Components

The second layer may further comprise 0-30% w/w of cellulose components, for example ethyl cellulose, hydroxypropyl cellulose or a mixture thereof. Suitably, 0-25% w/w of such components may be present.

In an embodiment, the second layer comprises 5 to 25% w/w of cellulose components.

In a further embodiment, the second layer comprises 5 to 25% w/w of an ethyl cellulose/hydroxypropyl cellulose mixture (e.g. at a ratio of 0.5-10:1 of ethyl cellulose:hydroxypropyl cellulose).

### Particular Embodiments of the Second Layer

The following represent particular embodiments of the second layer:

| | | | |
|---|---|---|---|
| 2.1 | 0.1-20% | w/w | opioid antagonist; |
| | 58-98% | w/w | pharmaceutically acceptable adhesive; |
| | 0-25% | w/w | cellulose components; |
| | 0-20% | w/w | hydrophilic material (e.g. propylene glycol or dipropylene glycol); |
| 2.2 | 8-12% | w/w | opioid antagonist; |
| | 60-70% | w/w | pharmaceutically acceptable adhesive (e.g. BIO-PSA 7-4302); |
| | 0-20% | w/w | cellulose components; |
| | 0-10% | w/w | hydrophilic material (e.g. propylene glycol or dipropylene glycol); |
| 2.3 | 0.1-10% | w/w | opioid antagonist selected from naltrexone (“NTX”), naloxone |
| | | | (“NLX”) or nalmefene, or a pharmaceutically acceptable salt |
| | | | thereof; |
| | 70-99% | w/w | pharmaceutically acceptable polyacrylate adhesive comprising a |
| | | | plurality of carboxy functional groups (e.g. Durotak-87-2054 ®, |
| | | | Durotak-87-2051 ®, Durotak-87-2052 ®, Durotak-87-2677 ®, Durotak- |
| | | | 87-2194 ®, Durotak-87-2196 ® or Durotak-87-2825 ®); |
| | 0-20% | w/w | hydrophilic material (e.g. propylene glycol or dipropylene glycol). |
| 2.4 | 0.1-5% | w/w | opioid antagonist selected from naltrexone (“NTX”), naloxone |
| | | | (“NLX”) or nalmefene, or a pharmaceutically acceptable salt thereof; |
| | 85-99% | w/w | pharmaceutically acceptable polyacrylate adhesive comprising a |
| | | | plurality of carboxy functional groups (e.g. Durotak-87-2054 ®, |
| | | | Durotak-87-2051 ®, Durotak-87-2052 ®, Durotak-87-2677 ®, Durotak- |
| | | | 87-2194 ®, Durotak-87-2196 ® or Durotak-87-2825 ®); |
| | 0-10% | w/w | hydrophilic material (e.g. propylene glycol or dipropylene glycol). |
| 2.5 | 0.1-5% | w/w | opioid antagonist selected from naltrexone (“NTX”), naloxone |
| | | | (“NLX”) or nalmefene, or a pharmaceutically acceptable salt thereof; |
| | 95-99% | w/w | pharmaceutically polyacrylate acceptable adhesive comprising a |
| | | | plurality of carboxy functional groups (e.g. Durotak-87-2054 ®, |
| | | | Durotak-87-2051 ®, Durotak-87-2052 ®, Durotak-87-2677 ®, Durotak- |
| | | | 87-2194 ®, Durotak-87-2196 ® or Durotak-87-2825 ®); |
| 2.6 | 1-3% | w/w | opioid antagonist selected from naltrexone (“NTX”), naloxone |
| | | | (“NLX”) or nalmefene, or a pharmaceutically acceptable salt thereof; |
| | 97-99% | w/w | pharmaceutically acceptable polyacrylate adhesive comprising a |
| | | | plurality of carboxy functional groups (e.g. Durotak-87-2054 ®, |
| | | | Durotak-87-2051 ®, Durotak-87-2052 ®, Durotak-87-2677 ®, Durotak- |
| | | | 87-2194 ®, Durotak-87-2196 ® or Durotak-87-2825 ®); |
| 2.7 | 1-3% | w/w | naltrexone (“NTX”), or a pharmaceutically acceptable salt thereof; |
| | 97-99% | w/w | pharmaceutically acceptable polyacrylate adhesive comprising a |
| | | | plurality of carboxy functional groups, an acrylate-vinyl acetate and |
| | | | a crosslinker, and wherein the pharmaceutically acceptable |
| | | | polyacrylate adhesive has a viscosity of between 2500 and 3500 mPa |
| | | | (e.g. Durotak-87-2054 ® or Durotak-87-2194 ®). |

Particular examples of the second layer are provided in the examples provided hereinbelow.

### Barrier Layer

The barrier layer is disposed between the first and second layers and functions to substantially prevent the opioid antagonist diffusing from the second layer to the first layer during normal use of the patch. The barrier layer also enables the release of the opioid antagonist from the second layer at a rate sufficient to inhibit the abuse potential of the opioid analgesic when the patch is tampered with (by, for example, chewing the patch or immersing it in a solvent such as water, ethanol or acetone).

In the first aspect of the present invention, a barrier layer comprising a water soluble cellulose derivative and a water insoluble cellulose derivative may be used.

In an embodiment, the barrier layer comprises between 10-30% of a water soluble cellulose derivative and between 70-90% of a water insoluble cellulose derivative, by weight on dry basis. Suitably, the barrier layer comprises between 15-25% of a water soluble cellulose derivative and between 75-85% of a water insoluble cellulose derivative, by weight on dry basis. Most suitably, the barrier layer comprises 20% of a water soluble cellulose derivative and 80% of a water insoluble cellulose derivative, by weight on dry basis.

In an embodiment, the water soluble cellulose derivative is selected from hydroxypropyl cellulose, hydroxypropyl methyl cellulose, hydroxyethyl cellulose, methyl cellulose, cellulose gum, sodium caboxymethylhydroxyethylcellulose, methyl hydroxycellulose or carboxymethyl cellulose. Suitably, the water soluble cellulose derivative is selected from hydroxypropyl cellulose, hydroxypropyl methyl cellulose, hydroxyethyl cellulose or methyl cellulose. Most suitably, the water soluble cellulose derivative is hydroxypropyl cellulose.

In an embodiment, the water insoluble cellulose derivative is selected from ethyl cellulose or cellulose ester. Suitably, the water insoluble cellulose derivative is ethyl cellulose.

Suitably, the barrier layer comprises hydroxypropyl cellulose and ethyl cellulose. More suitably, the barrier layer comprises between 15-25% hydroxypropyl cellulose and between 75-85% ethyl cellulose, by weight on a dry basis. Most suitably, the barrier layer comprises 20% hydroxypropyl cellulose and 80% ethyl cellulose, by weight on a dry basis

In an embodiment, the barrier layer is devoid of polyethylene glycol. Suitably, the barrier layer is devoid of polyethylene glycol and analogues thereof.

In another embodiment, the barrier layer is devoid of any additional hydrophilic polymers (e.g. polyethylene glycol, polyvinyl alcohol or polyacrylic acid). The additional hydrophilic polymer will be understood to be any hydrophilic (water soluble) polymer added in addition to the water soluble cellulose derivative and water insoluble cellulose derivative of the barrier layer.

Contrary to general common knowledge in the art, the omission of an additional hydrophilic polymer (e.g. polyethylene glycol) from the barrier layer was found to dramatically improve the processability of the barrier layer, whilst maintaining excellent barrier properties. Furthermore, through improving the processability of the barrier layer, greater control over barrier layer properties, such as thickness and the ability to prevent the opioid antagonist from diffusing from the second layer to the first layer, were found to be possible.

In another embodiment, greater than 80% w/w of the barrier layer is comprised of a water soluble cellulose derivative and a water insoluble cellulose derivative. Suitably, greater than 90% w/w of the barrier layer is comprised of a water soluble cellulose derivative and a water insoluble cellulose derivative. More suitably, greater than 95% w/w of the barrier layer is comprised of a water soluble cellulose derivative and a water insoluble cellulose derivative. Most suitably, greater than 99% w/w of the barrier layer is comprised of a water soluble cellulose derivative and a water insoluble cellulose derivative.

In an embodiment, the barrier layer consists essential of a water soluble cellulose derivative and a water insoluble cellulose derivative.

In another embodiment, the barrier layer consists of a water soluble cellulose derivative and a water insoluble cellulose derivative.

The barrier layer may be any suitable thickness. In an embodiment, the barrier layer has a thickness of between 0.1 and 4 mil (2.5 μm to 102 μm). Suitably, the barrier layer has a thickness of between 0.5 and 3 mil (12.5 μm to 76 μm). More suitably, the barrier layer has a thickness of between 1 and 2.5 mil (25 μm to 63.5 μm). Most suitably, the barrier layer has a thickness of between 1 and 2 mil (25 μm to 50 μm).

### Overlay

The transdermal patches of the present invention may optionally comprise an overlay layer to help adhere the patch to the skin. The overlay layer may be included for patches with a thickness of greater than about 0.75 mm, where extra adhesion to the skin may be necessary.

It will be appreciated that any suitable overlay (adhesive) may be used to adhere the patch to the skin. Suitably, the overlay is selected from a polyolefin, polyethylene or polyvinyl chloride foam tape.

In an embodiment, the overlay is a foam tape. Suitably, the overlay is a foam tape with one side coated with silicone.

In another embodiment, the overlay is a foam tape with a dry thickness of between 20 and 40 mil (0.51 mm to 1.02 mm). Suitably, the foam tape has a dry thickness of between 28 and 38 mil (0.71 mm to 0.97 mm), and most suitably between 30 and 35 mil (0.76 mm to 0.89 mm).

Suitably, the foam tape is coated with a liner (e.g. polyethylene coated paper). The liner may have any suitable thickness, with the liner typically having a thickness of 0.05% to 0.15% (e.g. 0.10%) of the thickness of the foam tape.

In an embodiment, the liner overlay liner has a thickness of between 2 to 10 mil (51 μm to 0.25 mm), suitably 4 to 6 mil (0.10 mm to 0.15 mm).

In another embodiment, the overlay is obtained from the company 3M.

In a further embodiment, the overlay is selected from 3M CoTran™ 9772L tape, 3M CoTran™ 9764 tape or 3M CoTran™ 9773 tape.

### Permeation Rate of Opioid Analgesic

It will be understood that the transdermal patch of the present invention, as defined herein, are formulated such that they are capable of administering the opioid analgesic transdermally during normal use. Accordingly, the transdermal patches of the present invention may be capable of permeating the opioid analgesic through the skin (i.e. human skin) during normal use, at a suitable permeation rate (otherwise known as flux).

In an embodiment, the transdermal patches of the present invention have an in vitro human skin permeation rate of opioid analgesic that is greater than 0.1 μg cm⁻² h⁻¹. The permeation of opioid analgesic through human skin has been measured for selected patches.

Permeation/release measurements of opioid analgesic through a human male skin (see Examples section hereinbelow) were used as a tool to select candidate patches.

By in vitro human skin permeation rate we mean the rate of delivery of opioid analgesic through human skin at time periods up to 72 hours.

Suitably, the in vitro human skin permeation rate of opioid analgesic is the apparent steady state flux (calculated from the approximately linear portion of the cumulative permeation profile), typically observed between 3 and 12 hours, or between 24 and 72 hours, when assessed under the conditions detailed in the following sections.

In an embodiment, the in vitro human skin permeation rate of opioid analgesic is between 0.1 μg cm⁻² h⁻¹ and 20 μg cm⁻² h⁻¹.

In a further embodiment, the in vitro human skin permeation rate of opioid analgesic is between 1.0 μg cm⁻² h⁻¹ and 10 μg cm⁻² h⁻¹.

In a further embodiment, the in vitro human skin permeation rate of opioid analgesic is between 2.5 μg cm⁻² h⁻¹ and 8.5 μg cm⁻² h⁻¹.

### Particular Embodiments of the Transdermal Patch

The following represent particular embodiments of the transdermal patch:

| 1.1 | First layer: | | |
|---|---|---|---|
| | 1-10% | w/w | opioid analgesic (e.g. oxymorphone or fentanyl); |
| | 58-98% | w/w | pharmaceutically acceptable pressure sensitive adhesive; |
| | 0.1-15% | w/w | penetration enhancer; |
| | 0-20% | w/w | hydrophilic material (e.g. propylene glycol). |
| | Barrier layer: | | |
| | 10-30% | w/w | water soluble cellulose derivative; |
| | 70-90% | w/w | water insoluble cellulose derivative; |
| | Second layer: | | |
| | 0.1-20% | w/w | opioid antagonist; |
| | 58-98% | w/w | pharmaceutically acceptable adhesive; |
| | 0-25% | w/w | cellulose components; |
| | 0-20% | w/w | hydrophilic material (e.g. propylene glycol or dipropylene glycol); |
| 1.2 | First layer: | | |
| | 1-10% | w/w | opioid analgesic (e.g. oxymorphone or fentanyl); |
| | 65-98% | w/w | pharmaceutically acceptable pressure sensitive polyacrylate |
| | | | adhesive comprising a plurality of hydroxyl functional groups (e.g. |
| | | | Durotak-87-4287 ®, Durotak-87-2287 ®, Durotak-87-2516 ® or |
| | | | Durotak-87-2525 ®); |
| | 0.1-15% | w/w | penetration enhancer selected from oleic acid or linoleic acid; |
| | 0-20% | w/w | hydrophilic material (e.g. propylene glycol); |
| | Barrier layer: | | |
| | 10-30% | w/w | water soluble cellulose derivative; |
| | 70-90% | w/w | water insoluble cellulose derivative; |
| | Second layer: | | |
| | 0.1-10% | w/w | opioid antagonist selected from naltrexone (“NTX”), naloxone |
| | | | (“NLX”) or nalmefene, or a pharmaceutically acceptable salt |
| | | | thereof; |
| | 70-99% | w/w | pharmaceutically acceptable polyacrylate adhesive comprising a |
| | | | plurality of carboxy functional groups (e.g. Durotak-87-2054 ®, |
| | | | Durotak-87-2051 ®, Durotak-87-2052 ®, Durotak-87-2677 ®, Durotak- |
| | | | 87-2194 ®, Durotak-87-2196 ® or Durotak-87-2825 ®); |
| | 0-20% | w/w | hydrophilic material (e.g. propylene glycol or dipropylene glycol); |
| 1.3 | First layer: | | |
| | 2-4% | w/w | opioid analgesic (e.g. oxymorphone or fentanyl); |
| | (e.g. 3% w/w) | | |
| | 91-95% | w/w | pharmaceutically acceptable pressure sensitive polyacrylate |
| | | | adhesive comprising a plurality of hydroxyl functional groups (e.g. |
| | | | Durotak-87-4287 ®, Durotak-87-2287 ®, Durotak-87-2516 ® or |
| | | | Durotak-87-2525 ®); |
| | 4-5% | w/w | penetration enhancer selected from oleic acid or linoleic acid); |
| | Barrier layer: | | |
| | 15-25% | w/w | water soluble cellulose derivative; |
| | 75-85% | w/w | water insoluble cellulose derivative; |
| | Second layer: | | |
| | 0.5-3 or 1-3% | w/w | opioid antagonist selected from naltrexone (“NTX”), naloxone |
| | | | (“NLX”) or nalmefene, or a pharmaceutically acceptable salt thereof; |
| | 97-99% | w/w | pharmaceutically acceptable polyacrylate adhesive comprising a |
| | | | plurality of carboxy functional groups (e.g. Durotak-87-2054 ®, |
| | | | Durotak-87-2051 ®, Durotak-87-2052 ®, Durotak-87-2677 ®, Durotak- |
| | | | 87-2194 ®, Durotak-87-2196 ® or Durotak-87-2825 ®); |
| 1.4 | First layer: | | |
| | 2-3% | w/w | opioid analgesic (e.g. oxymorphone or fentanyl); |
| | 92-95% | w/w | pharmaceutically acceptable pressure sensitive polyacrylate |
| | | | adhesive comprising a plurality of hydroxyl functional groups, an |
| | | | acrylate-vinyl acetate and a viscosity of between 7000 and 18000 mPa |
| | | | (e.g. Durotak-87-4287); |
| | 4-5% | w/w | penetration enhancer selected from oleic acid or linoleic acid); |
| | Barrier layer: | | |
| | 20% | w/w | water soluble cellulose derivative; |
| | 80% | w/w | water insoluble cellulose derivative; |
| | Second layer: | | |
| | 0.5-3 or 1-3% | w/w | naltrexone (“NTX”), or a pharmaceutically acceptable salt thereof; |
| | 97-99% | w/w | pharmaceutically acceptable polyacrylate adhesive comprising a |
| | | | plurality of carboxy functional groups, an acrylate-vinyl acetate and |
| | | | a crosslinker, and wherein the pharmaceutically acceptable |
| | | | polyacrylate adhesive has a viscosity of between 2500 and 3500 mPa |
| | | | (e.g. Durotak-87-2054 ® or Durotak-87-2194 ®). |

### Combination Patches

In one embodiment, the first layer of the particles defined herein containing the opioid analgesic can also be combined with an optional second non-opioid pharmacologically active agent for the treatment of pain and/or polydrug abuse, including, for example, a cannabinoid (agonist, antagonist, or inverse agonist), bupropion, hydroxybupropion, nicotine, nornicotine, varenicline, doxepin, acetaminophen, aspirin, diclofenac or another non-steroidal anti-inflammatory drug.

### Therapeutic Uses

The patches of the present invention may be used for the treatment of one or more medical conditions, such as opioid dependence, alcohol dependence, polydrug addiction, pain, cocaine addiction, eating disorders (e.g., binge eating) and treatment-resistant depression are described herein and comprise transdermally administering an opioid from an formulation as defined herein. In one embodiment, compositions described herein which are transdermally administrable include opioid analgesic (e.g. oxymorphone) and opioid antagonists, such as naltrexone and/or naloxone.

The compositions described herein are used in a “pharmacologically effective amount.” This means that the rate and extent of absorption of the active by the subject is such that it results in a therapeutic level of the active in the subject over the period that such compound is to be used. Such delivery is dependent on a number of variables including the time period for which the individual dosage unit is to be used, the flux rate of the active from the composition into the subject, for example, buprenorphine or a buprenorphine prodrug, from the formulation, surface area of the application site, etc.

In another embodiment, a single dosage unit comprises a therapeutically effective amount of opioid analgesic (e.g. oxymorphone). The term “therapeutically effective amount” or “therapeutically and/or prophylactically effective amount” as used herein refers to an amount of opioid analgesic that is sufficient to elicit the required or desired therapeutic and/or prophylactic response, as the particular treatment context may require. Single dosage unit as used herein includes individual patches. In one embodiment, a single dosage unit of any formulation comprises a therapeutically effective amount.

It will be understood that a therapeutically effective amount of opioid analgesic for a subject is dependent inter alia on the body weight of the subject as well as other factors known to a person of ordinary skill in the art. A “subject” herein to which a therapeutic agent or composition thereof can be administered includes mammals such as a human of either sex and of any age, and also includes any nonhuman animal, particularly a domestic, farm or companion animal, illustratively, a cat, cow, pig, dog or a horse as well as laboratory animals such as guinea pigs and primates.

In one embodiment, compositions described herein are suitable for transdermal administration. In another embodiment, transdermally administrable compositions are adapted for administration to the abdomen, back, chest, legs, arms, scalp or other suitable skin surface.

The terms “treat”, “treated”, “treating” and “treatment” are to be broadly understood as referring to any response to, or anticipation of, a medical condition in a mammal, particularly a human, and includes but is not limited to: (i) inhibiting the medical condition, i.e., arresting, slowing or delaying the on-set, development or progression of the medical condition; or (ii) relieving the medical condition, i.e., causing regression of the medical condition.

In one embodiment, a therapeutically effective amount of opioid analgesic is administered transdermally in an formulation as defined herein to treat a medical condition selected from the group consisting of: opioid dependence, alcohol dependence, polydrug addiction, pain, cocaine addiction, eating disorders (e.g., binge eating) and treatment-resistant depression.

Pain can include nociceptive pain, such as somatic pain and visceral pain, and non-nociceptive pain, such as neuropathic pain, sympathetic pain, psychogenic pain and idiopathic pain. Pain also includes chronic and acute pain. Non-limiting examples of pain or sources of pain include fibromyalgia, chronic back pain (both deep and superficial somatic pain), chronic pancreatitis, chronic acute hepatitis, gallstone, appendicitis, post-herpetic neuralgia, trigeminal neuralgia, phantom limb pain, diabetic neuropathy, carpal tunnel syndrome, sciatica, pudendal neuralgia, central pain syndrome, spinal cord injury, post-surgical pain, cancer, degenerative disk disease, osteoporosis, peripheral neuropathy, herpes zoster (shingles), lupus, reflex sympathetic dystrophy, headaches (migraines, tension and cluster), temporomandibular disorders, such as temporomandibular joint syndrome, myofacial pain and internal derangement of the joint and degenerative joint disease, such as osteoarthritis and rheumatoid arthritis.

Eating disorders can include anorexia nervosa, bulimia nervosa, binge eating disorder (BED), compulsive overeating, purging disorder, rumination, diabulimia, food maintenance, eating disorders not otherwise specified (EDNOS), pica, night eating syndrome and orthorexia nervosa.

In one embodiment, the pharmaceutical composition comprising an opioid analgesic, is administered once daily to a subject in need thereof. In a further embodiment, the pharmaceutical composition comprising an opioid analgesic, is administered twice daily to a subject in need thereof.

In another illustrative embodiment, a transdermal patch can be one which is capable of controlling the release of opioid analgesic or agonist-antagonists or prodrugs of the foregoing such that transdermal delivery of the active compound is substantially uniform and sustained over a period of about 6 hours, about 12 hours, about 24 hours, about 48 hours or about 7 days. Such transdermal patch which can be used in the practice of the methods described herein can take the form of an occlusive body having a backing layer. In practice, the occlusive body which includes the opioid agonists or agonist-antagonists or prodrugs of the foregoing is positioned on the subject's skin under conditions suitable for transdermally delivering the active compound to the subject

### Preparation of Pharmaceutical Formulations

The transdermal patches of the present invention can be prepared using conventional techniques known in the art.

The first and second layers defined herein are suitably prepared by mixing all of the components together to form first and second layer compositions respectively, which may then be cast onto a suitable surface (e.g. release liner). The individual components may be mixed by simply adding all of the components at the same time into a mixing vessel and then mixing them all together (a “one-pot” mixture). Alternatively, the components may be added sequentially in two or more steps or stages.

Other experimental conditions required to prepare the formulations of the present invention, such as mixing times, mixing equipment, temperature control etc. can be readily determined by a person of ordinary skill in the art.

In an embodiment, the first layer composition is prepared by the following process:
(i) the pressure sensitive polyacrylate adhesive is mixed with a suitable organic solvent (e.g. ethyl acetate);
(ii) opioid analgesic (e.g. oxymorphone) is slowly added to the solution of step (i), with mixing;
(iii) the penetration enhancer is added to the solution of step (ii), with mixing; and
(iv) the solution of step (iii) is further mixed using a roller mixer for at least 8 hours.

Suitably, the mixture of step (iii) is mixed on a roller mixture for at least 10 hours, more suitably, for at least 12 hours, and most suitably for at least 15 hours.

In another embodiment, the second layer composition is prepared by the following process:
a) the pressure sensitive polyacrylate adhesive is mixed with a suitable organic solvent (e.g. ethyl acetate);
b) the opioid antagonist is slowly added to the solution of step a), with mixing;
c) the solution of step b) is further mixed using a roller mixer for at least 8 hours.

Suitably, the mixture of step b) is mixed on a roller mixture for at least 10 hours, more suitably, for at least 12 hours, and most suitably for at least 15 hours.

Further experimental details will also be evident from the accompanying Examples.

The barrier layer defined herein may be suitably prepared by mixing all of the components together in the presence of an organic solvent (e.g. ethanol or ethyl acetate) so as to form a barrier layer composition.

In an embodiment the barrier layer composition is prepared by the following process:
1) the water soluble cellulose derivative (e.g. hydroxypropyl cellulose) is slowly added to an organic solvent (e.g. ethanol) with mixing, to form a solution of water soluble cellulose derivative;
2) the water insoluble cellulose derivative (e.g. ethyl cellulose) is slowly added to an organic solvent (e.g. ethyl acetate) with mixing, to form a solution of water insoluble cellulose derivative;
3) the solution of water soluble cellulose derivative of step 1 is slowly added to a pre-weighed amount of the solution of water insoluble cellulose derivative of step 2, with mixing, to form a clear solution of the barrier layer composition.

In an embodiment, the organic solvent of step 1 is ethanol.

In another embodiment, the organic solvent of step 2 is devoid of any chloroform.

Suitably, the organic step of step 2 is ethyl acetate.

In an embodiment, the solution of water soluble cellulose derivative of step 1 is a 5-30% w/w solution, suitably a 10-20% w/w solution, and more suitably a 15% w/w solution.

In an embodiment, the solution of water insoluble cellulose derivative of step 2 is a 5-30% w/w solution, suitably a 10-20% w/w solution, and more suitably a 15% w/w solution.

In another embodiment, the solution of water soluble cellulose derivative of step 1 has a viscosity, at room temperature, of between 1000 and 3000 cps, suitably between 1500 and 2500 cps, and most suitably between 1800 and 2200 cps.

In an embodiment, the solution of water insoluble cellulose derivative of step 2 has a viscosity, at room temperature, of between 10000 and 16000 cps, suitably between 12000 and 15000 cps, and most suitably between 13000 and 14000 cps.

In an embodiment, the barrier layer composition comprises between 15-25% (e.g. 20%) of the solution of water soluble cellulose derivative and between 75-85% (e.g. 80%) of the solution of water insoluble cellulose derivative, by weight.

In a further embodiment, the barrier layer composition of step 3 has a viscosity, at room temperature, of between 3000 and 7000 cps, suitably between 4000 and 6000 cps, and most suitably between 4250 and 4750 cps.

Once components have been mixed together the layers can be prepared by wet casting a desired thickness of each composition onto a suitable surface, e.g. a release liner. The layer can then be dried and stored ready for assembly.

Typically, the first and second layer compositions are cast at a wet thickness of between 10-60 mil (254 μm to 1.52 mm), to provide a dry thickness of between 4 and 12 mil (102 μm and about 305 μm), suitably between 5 and 11 mil (127 μm and about 279 μm). After casting, the layers are dried.

The first and second layer compositions may be roller cast onto a suitable surface (e.g. release liner) using any suitable coating gap, i.e. the gap set between rollers. Suitably, the coating gap is set to between 0.100 and 3.00 mm. More suitable, the coating gap is set to between 0.100 and 1.50 mm. Yet more suitably, the coating gap is set to between 0.100 and 1.200 mm. Even more suitably, the coating gap is set to between 0.100 and 0.700 mm. Most suitably, the coating gap is set to between 0.300 and 0.700 mm.

In an embodiment, the barrier layer may be roller cast onto a suitable surface (e.g. release liner) using any suitable coating gap of between 0.100 and 3.00 mm, suitably between 0.100 and 1.00 mm, more suitably between 0.200 mm and 0.800 mm, and most suitably between 0.300 and 0.600 mm.

The layers are assembled to form the multilaminate structures defined herein. Suitably, the first layer composition is wet cast onto a release liner as defined herein (e.g. 3M Scotchpak 9744). One surface of the first layer is therefore in contact with the release liner and the barrier layer is applied to the opposing surface, followed by the second layer and a barrier layer to complete the assembly of the patch.

In an embodiment, the transdermal patches of the present invention may be prepared according the following procedure:
1) Coat the second layer composition on to a release liner (e.g. 3M Scotchpak 9744) using a coating gap of between 0.100 and 3.00 mm;
2) Dry the coated release liner of step 1);
3) Laminate the coated release liner of step 2) with a backing film (e.g. 3M Scotchpak 9733);
4) Coat the barrier layer composition on to a release liner (e.g. 3M Scotchpak 9744) using a coating gap of between 0.100 and 3.00 mm;
5) Dry the coated release liner of step 4);
6) Laminate the coated release liner of step 5) with a release liner (e.g. 3M Scotchpak 9744);
7) Coat the first layer composition on to a release liner (e.g. 3M Scotchpak 9744), using a coating gap of between 0.100 and 3.00 mm;
8) Dry the coated release liner of step 7);
9) Laminate the coated release liner of step 8) with a release liner (e.g. 3M Scotchpak 9744);
10) Remove the release liner from the second layer laminate (e.g. the laminate of step 3) as well as one of the release liner from the barrier layer laminate (e.g. the laminate of step 6);
11) Laminate the second layer laminate and barrier layer laminate together i.e. the laminates of steps 3 and 6;
12) Remove the release liner from the first layer laminate (e.g. the laminate of step 9);
13) Remove release liner from the combined layers of step 11;
14) Laminate the combined second layer laminate and barrier layer laminate with the first layer laminate i.e. the laminates of steps 9 and 11;
15) Cut patches of appropriate sizes from the resulting laminate.

It will be understood that the order of the steps described hereinabove for the preparation of the transdermal patches may be varied. For example, you could prepare the first, second and barrier layers in any suitable order, and assemble them in any order, so long as the transdermal patch comprises a first and second layer separated by a barrier layer.

In order to achieve thicker layers of the transdermal patches of the present invention, and thus more of the associated component (e.g. oxymorphone), each layer may be coated multiple times.

In an embodiment, the transdermal patches of the present invention are prepared by coating the first, second and backing layers onto the associated surface one or more times.

The transdermal patches of the present invention may be obtained, directly obtained, or obtainable by a process as described hereinabove.

### EXAMPLES

### Description of Drawings

Embodiments of the invention will be described, by way of example only, with reference to the accompanying drawings, in which:

FIG. 1 shows a plot of the average (n=5) cumulative amount of Oxymorphone (μg/cm²) permeated over time for a 2.5% Oxymorphone transdermal system comprising 2.5% Linoleic Acid (1.2 mm, 10.6 mil, 6.83 mg, 10.75 cm², the patch of Table 6, entry 1 hereinbelow) permeated in vitro through human cadaver skin.

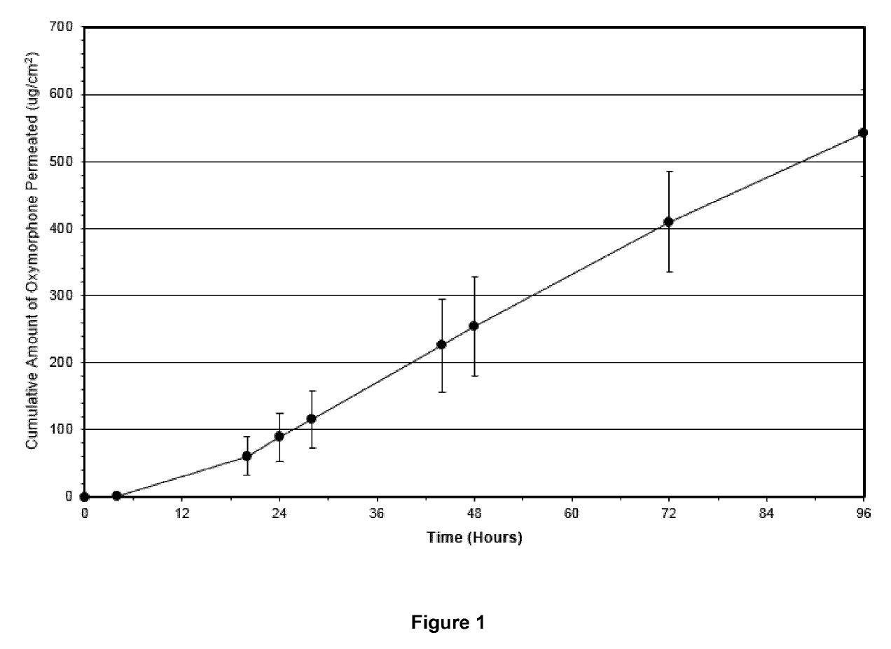

FIG. 2 shows a plot of the average (n=5) Oxymorphone Permeation Rate (μg/cm²/h) over time for a 2.5% Oxymorphone transdermal system comprising 2.5% Linoleic Acid (1.2 mm, 10.6 mil, 6.83 mg, 10.75 cm², the patch of Table 6, entry 1 hereinbelow) permeated in vitro through human cadaver skin.

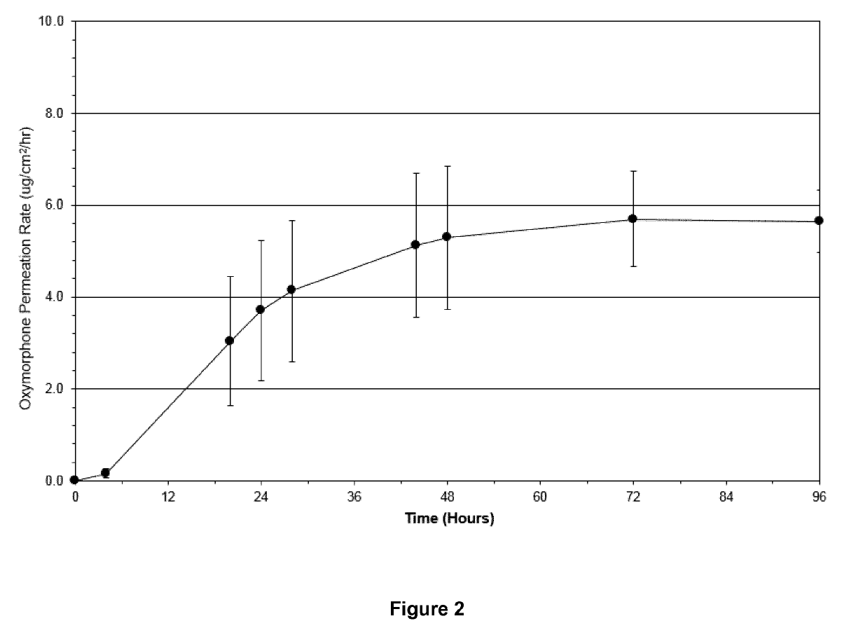

FIG. 3 shows a plot of the average (n=5) cumulative amount of Naltrexone (μg/cm²) permeated over time for a 3.0% Naltrexone transdermal system (1.0 mm, 12.4 mil, 9.12 mg/patch, 10.75 cm², the patch of Table 8 hereinbelow) permeated in vitro through human cadaver skin.

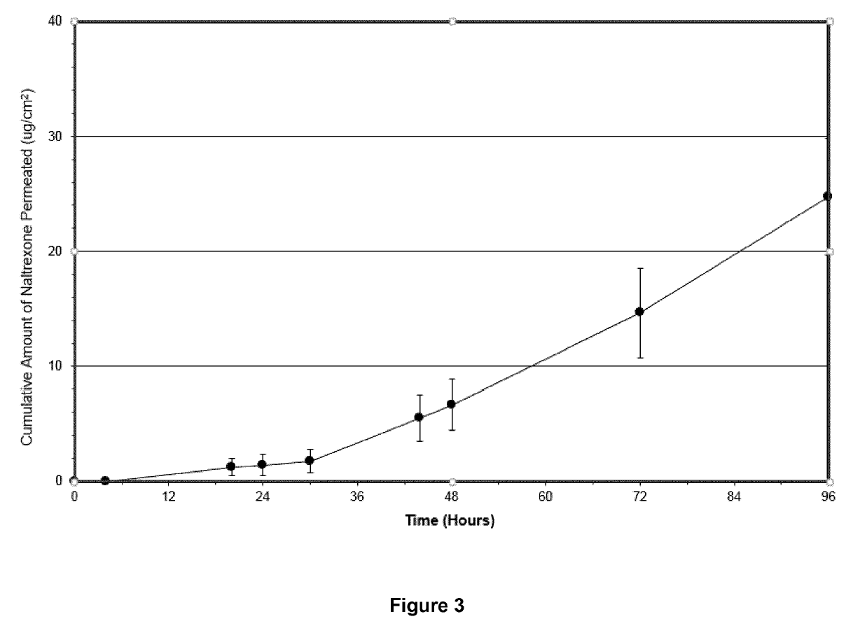

FIG. 4 shows a plot of the average (n=5) Naltrexone Permeation Rate (μg/cm²/h) over time for a 3.0% Naltrexone transdermal system (1.0 mm, 12.4 mil, 9.12 mg/patch, 10.75 cm², the patch of Table 8 hereinbelow) permeated in vitro through human cadaver skin.

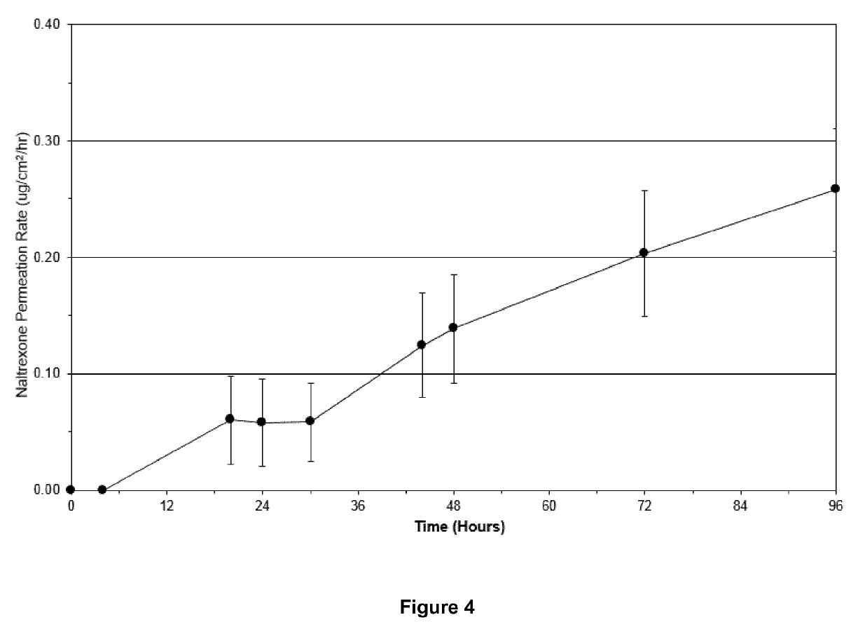

FIG. 5 shows a plot of the average (n=5) cumulative amount of Oxymorphone (μg/cm²) permeated over time for a multilaminate patch comprising 3.0% Naltrexone, an EC-HPC barrier layer and 2.5% Oxymorphone (the patch of Table 9 hereinbelow). The graph shows the in vitro Oxymorphone permeation through human cadaver skin.

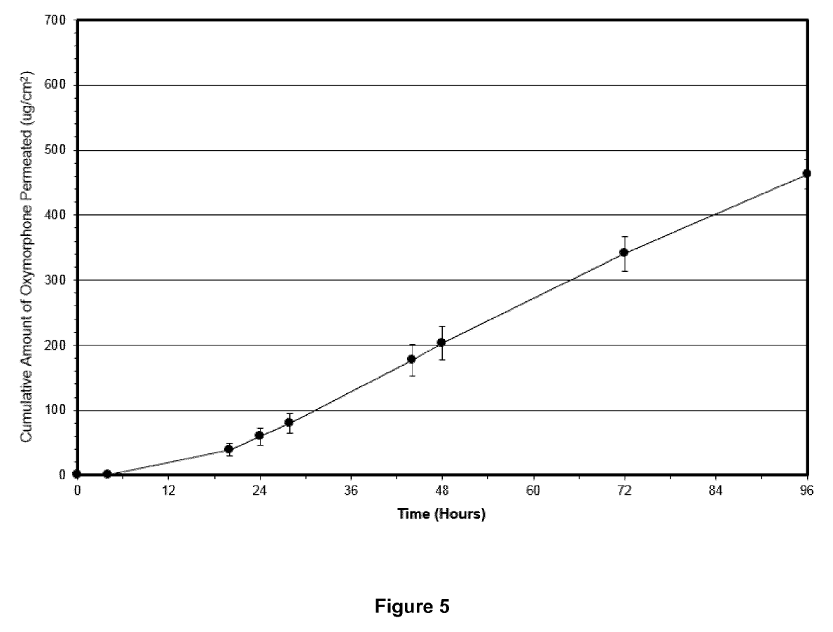

FIG. 6 shows a plot of the average (n=5) Oxymorphone Permeation Rate (μg/cm²/h) over time for a multilaminate patch comprising 3.0% Naltrexone, an EC-HPC barrier layer and 2.5% Oxymorphone (the patch of Table 9 hereinbelow). The Oxymorphone Permeation Rate is determined through the in vitro permeation of Oxymorphone through human cadaver skin.

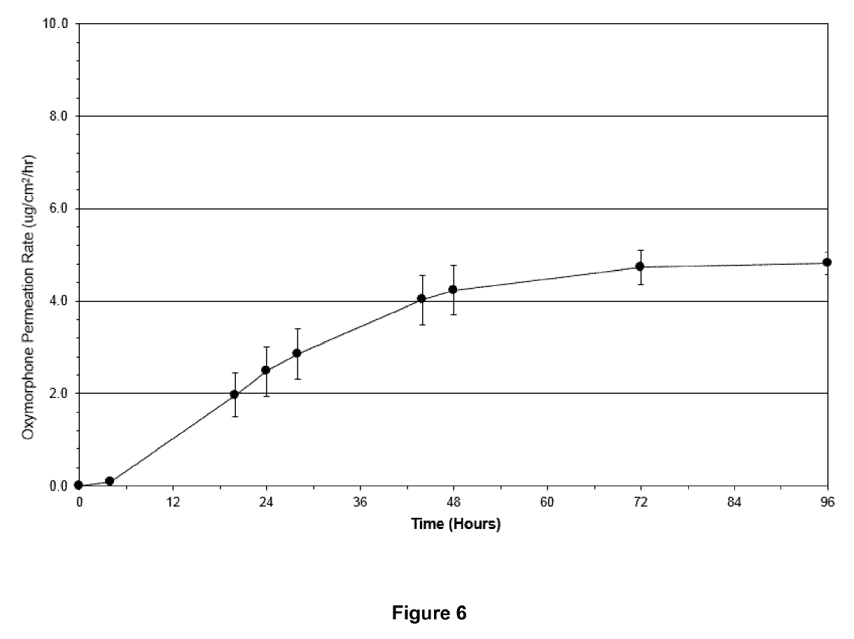

FIG. 7 shows a schematic depicting a typical assembly of a transdermal patch of the present invention (in which: 1 is the backing membrane; 2 is the first layer comprising the oxymorphone; and 3 is the release liner; 4 is the second layer comprising the opioid antagonist; and 5 is the barrier layer).

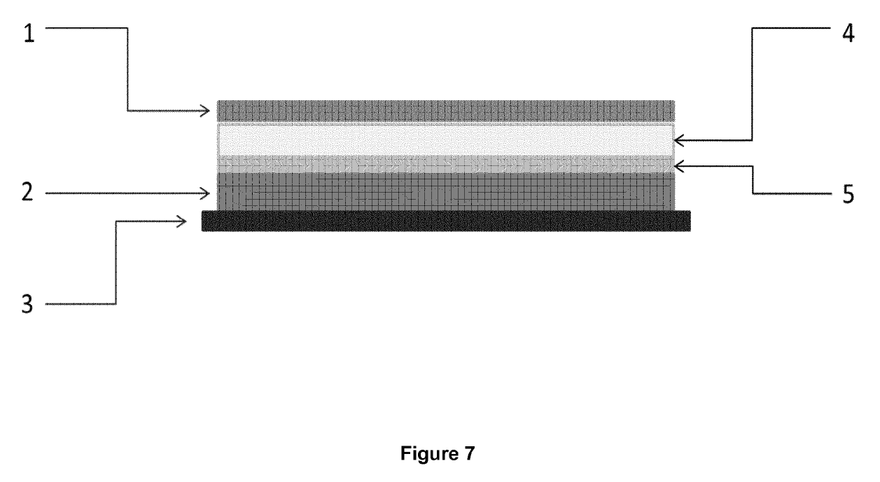

### Materials and Procedures

### Chemicals

The various chemicals used throughout these examples are as follows:

| Chemical | Manufacturer | Lot/Batch | CAS |
|---|---|---|---|
| Oxymorphone base | Mallinckrodt Inc. | 1310000735 | 76-41-5 |
| Duro-Tak 87-4287 ® (PSA) | Henkel Corporation | ZQ62566751 | |
| Linoleic acid | Spectrum | ZR1141 | 60-33-3 |
| Oleic acid | Croda | 896947 | 112-80-1 |
| Ethyl acetate | | 2DE0179 | 141-78-6 |
| Klucel EF PHARM - hydroxy propyl | Ashland Speciality | 52565 | Product code: |
| cellulose | Ingredients | | 431216 |
| Aqualon EC-N50 PHARM - ethyl cellulose | Ashland Speciality | 43922 | Product code: |
| | Ingredients | | 424925 |
| Ethyl alcohol, 190 proof | Pride Solvents and | M27980M | 64-17-5 |
| | Chemical | | |
| Duro-Ta87-2054 (PSA) | Henkel Corporation | CHY-166 | |
| | (National Starch) | | |
| Naltrexone base | Mallinckrodt | 14080000108 | 16590-41-3 |

### Procedures

### Permeation Studies

### Skin Permeation

Dermatomed/split thickness skin dermatomed to a thickness of about 375 μm from leg (left posterior leg or right posterior leg) was received from Skin Bank. Skin was stored at −5° C. until used for the permeation studies.

### Permeation Studies

Franz Diffusion Cells were used for the skin permeation studies, with the following protocol used.

Diffusion cells were kept at 37° C. with a heated circulating water bath. Skin was cut 3 cm×3 cm and mounted on the top of the receptor of the Franz cells facing stratum corneum/epidermis layer up and dermis layer facing down towards the receptor. The release liner was removed from the transdermal patch (10.5 cm² or 10.75 cm²) and the exposed adhesive layer was applied to the stratum corneum layer with slight pressure. The donor cap was clamped to the receptor compartment. The permeation area of the skin was 1.767 cm². The samples were collected from 3 to 5 cells per formulation. The receptor/receiver solution was PBS (phosphate buffer saline) pH 7.4 solution. Entire samples were withdrawn from the receptor compartment at predetermined time points at 4, 20, 24, 28, 44, 48, 72 and 96 hours and replaced the same volume with fresh PBS solution. The samples were placed screw cap test tubes and refrigerated until ready for HPLC analysis.

### Extraction Studies

### In Ethanol:

Oxymorphone or Naltrexone patches (10.5 cm²) were placed on a polypropylene mesh and placed in a 250 mL glass stoppered conical flask with 100 mL of ethanol. The patches were extracted for 24 hours and the amount of Oxymorphone or Naltrexone was determined, typically by HPLC.

### In pH 6.3 Buffer:

Oxymorphone or Naltrexone patches (10.5 cm²) were placed on a polypropylene mesh and placed in a 250 mL glass stoppered conical flask with 100 mL of pH 6.3 phosphate buffer. The patches were extracted for 24 hours and the amount of Oxymorphone or Naltrexone was determined, typically by HPLC.

### Results

### First Layer

### Adhesive

Adhesive transdermal patches were firstly prepared using 3% oxymorphone in various adhesives and determined the corresponding flux values through Male skin age 45 (skin bank JL102114) using Phosphate buffered saline at pH of 7.4. The corresponding flux values are detailed in Table 1 below.

TABLE 1
Flux values of Oxymorphone through Male skin for
transdermal patches comprising various adhesives

| | | | | | | Flux - t |
|---|---|---|---|---|---|---|
| Experiment | Adhesive | Adhesive | Functional | Vinyl | Cross | 24 to 72 |
| Number | Used | Type | Group | Acetate | Linker | μg/cm²/h |
| 1 | Duro-Tak | Polyacrylate | None | VA | None | 2.72 ± 0.42 |
| | 87-4098 | | | Present | | |
| 2 | Duro-Tak | Polyacrylate | Hydroxyl | VA | Present | 4.69 ± 0.44 |
| | 87-2516 | | (—OH) | Present | | |
| 3 | Duro-Tak | Polyacrylate | Carboxyl | VA | None | 0.11 ± 0.03 |
| | 87-2051 | | (—COOH) | Present | | |
| 4 | Duro-Tak | Polyacrylate | Carboxyl | VA | Present | 0.36 ± 0.12 |
| | 87-2054 | | (—COOH) | Present | | |
| 5 | Duro-Tak | Polyacrylate | Hydroxyl | None | None | 2.66 ± 0.42 |
| | 87-2510 | | (—OH) | | | |
| 6 | Duro-Tak | Polyacrylate | None | None | None | 1.65 ± 0.27 |
| | 87-9088 | | | | | |
| 7 | Duro-Tak | Polyacrylate | Carboxyl | VA | Present | 0.15± 0.05 |
| | 87-2194 | | (—COOH) | Present | | |
| 8 | Duro-Tak | Polyacrylate | Hydroxyl | VA | None | 3.74 ± 0.23 |
| | 87-4287 | | (—OH) | Present | | |
| 9 | BIO-PSA | Silicone | | | | 0.78 ± 0.11 |
| | 7-4302 | | | | | |
| 10 | Silac Hybrid | Silicone + | | | | 1.23 ± 0.21 |
| | 7-6302 | Polyacrylate | | | | |
| 11 | Duro-Tak | Polyisobutylene | | | | 0.05 ± 0.02 |
| | 87-6918 | | | | | |

Table 1 illustrates the dramatic and unexpected increase in Oxymorphone flux in using polyacrylate adhesives comprising a plurality of hydroxyl functional groups.

### Penetration Enhancer

Next adhesive transdermal patches comprising oxymorphone (3% w/w) in Durotak 87-4287 adhesive were prepared using 5 different penetration enhancers (5% w/w) (Oleic acid; oleyl alcohol; oleyl alcohol; lauryl lactate; lauryl alcohol and linoleic acid). Also prepared was a transdermal patch containing oxymorphone (3% w/w) in Durotak 87-2516 adhesive only. Flux values through Male white skin age 62 (skin bank KMG032615) using Phosphate buffered saline at pH of 7.4 were then determined. The corresponding flux values are in Table 2 below.

It was shown that in all cases, the addition of a penetration enhancer increased Oxymorphone flux through the skin. Penetration enhancers linoleic acid and oleic acid were shown to give substantially increased Oxymorphone flux.

TABLE 2
Flux values of Oxymorphone through Male skin for transdermal
patches comprising various penetration enhancers

| | | | | | | Flux - t |
|---|---|---|---|---|---|---|
| Adhesive | Adhesive | Functional | Vinyl | Cross | Penetration | 24 to 72 |
| Used | Type | Group | Acetate | Linker | Enhancer | μg/cm²/h |
| Durotak | Polyacrylate | Hydroxyl | Present | No | None | 3.15 ± 0.88 |
| 87-4287 | | | | | | |
| Durotak | Polyacrylate | Hydroxyl | Present | No | Oleic Acid | 4.21 ± 0.68 |
| 87-4287 | | | | | | |
| Durotak | Polyacrylate | Hydroxyl | Present | No | Oleyl Alcohol | 2.33 ± 0.66 |
| 87-4287 | | | | | | |
| Durotak | Polyacrylate | Hydroxyl | Present | No | Lauryl Lactate | 2.27 ± 0.48 |
| 87-4287 | | | | | | |
| Durotak | Polyacrylate | Hydroxyl | Present | No | Lauryl Alcohol | 3.53 ± 0.19 |
| 87-4287 | | | | | | |
| Durotak | Polyacrylate | Hydroxyl | Present | No | Linoleic Acid | 3.62 ± 0.76 |
| 87-4287 | | | | | | |

### Second Layer

### Adhesive

Adhesive transdermal patches were made using naltrexone free base in various adhesives and the corresponding flux values through White Male skin age 62 (skin bank MG032615) using Phosphate buffered saline at pH of 7.4 were determined. The corresponding flux values are in Table 3 below.

The results in Table 3 show that polyacylate adhesives comprising carboxy functional groups demonstrated the lowest Naltrexone flux.

TABLE 3
Flux values of Naltrexone through Male skin for
transdermal patches comprising various adhesive

| Amount of | | | | | | Flux - t |
|---|---|---|---|---|---|---|
| Naltrexone | Adhesive | Adhesive | Functional | Vinyl | Cross | 24 to 72 |
| (mg) | Used | Type | Group | Acetate | Linker | (μg/cm²/h) |
| 2.69 mg | Duro-Tak | Polyacrylate | None | VA | None | 2.30 ± 0.39 |
| | 87-4098 | | | Present | | |
| 2.87 mg | Duro-Tak | Polyacrylate | Hydroxyl | VA | Present | 3.00 ± 0.86 |
| | 87-2516 | | (—OH) | Present | | |
| 2.48 mg | Duro-Tak | Polyacrylate | Hydroxyl | VA | None | 2.74 ± 0.78 |
| | 87-4287 | | (—OH) | Present | | |
| 3.41 mg | Duro-Tak | Polyacrylate | Carboxyl | VA | Present | 1.22 ± 0.57 |
| | 87-2054 | | (—COOH) | Present | | |
| 5.83 mg | BIO-PSA | Silicone | | | | 2.67 ± 0.12 |
| | 7-4302 | | | | | |
| 1.99 mg | Duro-Tak | Polyisobutylene | | | | 1.29 ± 0.22 |
| | 87-6918 | | | | | |

### Single Layer Formulations

Next Oxymorphone and Naltrexone flux and extraction in ethanol was determined for the first and second layers respectively. The results are summarised in Table 4 below.

TABLE 4
The extraction and flux data for optimised first and second layers

| | | | OXYMORPHONE (OXY) |
|---|---|---|---|
| | | | FLUX or naltrexone flux |
| | | | determined using male white |
| | Coating | | skin age 62 Section RPL |
| Formulation | gap | Extraction Data | (μg/cm²/h) |
| 3% | 0.600 mm | OXYMORPHONE ASSAY | Average flux 24-72 hrs = 6.52 |
| oxymorphone | | 1^{st} extraction in ethanol = 4.20 mg | |
| in Durotak | | 2^{nd} extraction in ethanol = 0.12 mg | |
| 87-4287 and | | total mg/patch = 4.32 mg | |
| 5% oleic | | 1^{st} extraction in buffer pH 6.3 = 1.72 | |
| acid | | 2^{nd} extraction in ethanol = 2.17 mg | |
| | | total mg/patch = 3.89 mg | |
| 3% | 0.600 mm | OXYMORPHONE ASSAY | Average flux 24-72 hrs = 6.96 |
| oxymorphone | | 1^{st} extraction in ethanol = 4.15 mg | Average flux 0-72 hrs = 6.27 |
| in Durotak | | 2^{nd} extraction in ethanol = 0.12 mg | |
| 87-4287 and | | total mg/patch = 4.27 mg | |
| 5% linoleic | | 1^{st} extraction in buffer pH 6.3 = 1.65 mg | |
| acid | | 2^{nd} extraction in ethanol = 2.01 mg | |
| | | total mg/patch = 3.66 mg | |
| 1% | 0.400 mm | NALTREXONE ASSAY | Average flux naltrexone 24-72 hrs = |
| Naltrexone in | | 1^{st} extraction in ethanol = 1.13 mg | 0.09 |
| Durotak 87- | | 2^{nd} extraction in ethanol = 0.02 mg | |
| 2054 | | total mg/patch = 1.15 mg | |
| | | 1^{st} extraction in buffer pH 6.3 = 0.19 mg | |
| | | 2^{nd} extraction in ethanol = 0.93 mg | |
| | | total mg/patch = 1.12 mg | |
| 3% | 0.4 mm | NALTREXONE ASSAY | Average flux naltrexone 24-72 hrs = |
| Naltrexone in | | 1^{st} extraction in ethanol = 3.42 mg | 0.50 |
| Durotak 87- | | 2^{nd} extraction in ethanol = 0.07 | |
| 2054 | | total mg/patch = 3.49 mg | |
| | | 1^{st} extraction in buffer pH 6.3 = 0.59 mg | |
| | | 2^{nd} extraction in ethanol = 2.84 mg | |
| | | total mg/patch = 3.43 mg | |

The results in Table 4 show that for the first layer an excellent level of Oxymorphone flux is observed when both linoleic acid and oleic acid are used as the penetration enhancer.

Whereas, for the second layer a low level of Naltrexone flux is observed for patches containing both 1% and 3% of Naltrexone.

The extraction data presented in Table 4 shows that excellent levels of Oxymorphone and Naltrexone are extracted for first and second layers respectively.

Oxymorphone flux was then determined for the first layer of the transdermal patches of the present invention at different coating thicknesses. The flux value are summarised in Tables 5 and 6 below.

TABLE 5
Flux values of Oxymorphone for transdermal patches comprising 3% Oxymorphone at
different coating thicknesses.

| OXYMORPHONE | | Coating | Permeation | | |
|---|---|---|---|---|---|
| Content | Durotak | gap | enhancer | Skin Bank donor | Flux (t24-t72 hrs) |
| 3% | 87-4287 | 0.400 | Linoleic | M W (62) | 3.62 +/− 0.76 |
| | | | acid (5%) | MG032615) LPL | |
| 3% | 87-4287 | 0.600 | Linoleic | M W (62) | 6.96 |
| | | | acid (5%) | MG032615 RPL | |
| 3% | 87-4287 | 0.600 | Linoleic | M W (69) | 6.98 |
| | | | acid (5%) | MM041115 | |
| | | | | PT | |

TABLE 6
Further flux values of Oxymorphone for transdermal patches comprising 3%
Oxymorphone at different coating thicknesses.

| | | | FLUX VALUE | |
|---|---|---|---|---|
| | Amount of | COATING | (t 24-72 hrs; | |
| | Oxymorphone | GAP | units: | |
| % OXYMORPHONE | per cm² | (mm) | μg/cm²/h) | COMMENT |
| 3% oxymorphone in | 4.10 mg/10.5 cm² | 5.4 mil | 7.22 (a) | SKIN |
| Durotak 87-4287 and | (15.6 mg/40 mg²) | (0.6 mm) | 6.74 (b) | MM040002 |
| 5% linoleic acid | 390 μg/cm² | | 7.11 (c) | Age 69 |
| | | | 6.88 (d) | section pt |
| | | | 6.83 (e) | |
| | | | Average of 5 | |
| | | | 6.95 μg/cm²/hr | |
| 3% oxymorphone in | 4.24 mg/10.5 cm² | 5.1 mil | 6.81(a) | SKIN |
| Durotak 87-4287 and | (15.77 mg/40 mg²) | (0.6 mm) | 6.72 (b) | JA040115 |
| 5% linoleic acid | 404 μg/cm² | | 4.03 (c) | Age 67 LPL |
| | | | Average of 3 | |
| | | | 5.86 μg/cm²/hr | |
| 3% oxymorphone in | 5.32 mg/ | 6.3 mil | 7.14(a) | SKIN |
| Durotak 87-4287 and | 10.75 cm² | (0.8 mm) | 6.09 (b) | JA040115 |
| 5% linoleic acid | (19.91 mg/40 mg²) | | 7.59 (c) | Age 67 LPL |
| | 498 μg/cm² | | Average of 3 | |
| | | | 6.94 μg/cm²/hr | |
| 3% oxymorphone in | 6.87 mg/ | 8.1 mil | 7.17(a) | SKIN |
| Durotak 87-4287 and | 10.75 cm² | (1.0 mm) | 7.83 (b) | JA040115 |
| 5% linoleic acid | (25.56 mg/40 mg²) | | 7.60 (c) | Age 67 LPL |
| | 639 μg/cm² | | Average of 3 | |
| | | | 7.54 μg/cm²/hr | |

### Effects of the Penetration Enhancer

Comparison of patches with and without penetration enhancer were next prepared. The results are summarised in Table 7 below.

TABLE 7
Flux values of Oxymorphone for transdermal patches comprising either no penetration
enhancer or 5% w/w linoleic acid at different coating thicknesses.

| | Amount of | COATING | FLUX VALUE | |
|---|---|---|---|---|
| | Oxymorphone | GAP | (t 24-72 hrs; units: | |
| % OXYMORPHONE | per cm² | (mm) | μg/cm²/h) | COMMENT |
| 3% oxymorphone in | 3.14 mg/10.5 cm² | 3.7 mil (0.4 mm) | 3.85 (a) | SKIN |
| Durotak 87-4287 | (11.96 mg/40 mg²) | | 3.42 (b) | MG032615 |
| | 299 μg/cm² | | 2.16 (c) | Age 62 (white) |
| | | | Average of 3 | section LPL |
| | | | 3.14 μg/cm²/hr | |
| 3% oxymorphone in | 2.93 mg/10.5 cm² | 4.2 mil (0.4 mm) | 2.79 (a) | SKIN |
| Durotak 87-4287 and 5% | (11.16 mg/40 mg²) | | 3.78 (b) | MG032615 |
| linoleic acid | 279 μg/cm² | | 4.28 (c) | Age 62 LPL |
| | | | Average of 3 | |
| | | | 3.62 μg/cm²/hr | |
| 3% oxymorphone in | 4.27 mg/10.5 cm² | 0.6 mm | 7.04 (a) | SKIN |
| Durotak 87-4287 and 5% | (16.27 mg/40 mg²) | | 7.74 (b) | MG032615 |
| linoleic acid | 407 μg/cm² | | 7.07 (c) | Age 62 (white) |
| | | | 6.98 (d) | section RPL |
| | | | 6.48 (e) | |
| | | | Average of 5 | |
| | | | 6.96 μg/cm²/hr | |
| 3% oxymorphone in | 4.12 mg/10.5 cm² | 5.4 mil (0.6 mm) | 7.22 (a) | SKIN |
| Durotak 87-4287 and 5% | (15.61 mg/40 mg²) | | 6.74 (b) | MM041115 |
| linoleic acid | 392 μg/cm² | | 7.11 (c) | Age 69 section |
| | | | 6.88 (d) | pt |
| | | | 6.83 (e) | |
| | | | Average of 5 | |
| | | | 6.95 μg/cm²/hr | |

### Multilaminate Transdermal Patches

### Manufacturing Process: Prototype Patch

The transdermal patches of the present invention may be prepared as described in the prototype procedure below:

### Equipment Used

Coating Equipment Used: Werner-Mathis Lab Coater, Coater—Model LTSV, S/N 75288, Drying Oven—Model LTF, S/N124188
Air Distribution Setting—Middle, Position 0, Amount Air—Water Level Difference, Increments 10, Fan Revolutions—2250 rpm

### Procedure

(1) Coat 3% Naltrexone in Acrylate Adhesive DT 87-2054, Lot# N091115-K on Release Liner 3M Scotchpak 9744, 3.0 mil at 0.400 mm gap between knife and roll.
(2) Dry at 60° C. for 10 minutes.
(3) Laminate the coated release liner layer with backing film 3M Scotchpak 9733, 2.0 mil
(4) Apply 4-pound roller 2 times for better contact.
(5) Coat Barrier Layer 80% Ethyl Cellulose+20% Hydroxy Propyl Cellulose, Lot#081915-EC+HPC on Release Liner 3M Scotchpak 9744, 3.0 mil at 0.400 mm gap between knife and roll
(6) Dry at 60° C. for 10 minutes.
(7) Laminate the coated release liner layer with 3M Scotchpak 9744, 3.0 mil release liner.
(8) Apply 4-pound roller 2 times for better contact.
(9) Coat 3% Oxymorphone in Acrylate Adhesive DT 87-4287+5% Linoleic Acid, Lot#O072415-AA on Release Liner 3M Scotchpak 9744, 3.0 mil at 0.600 mm gap between knife and roll.
(10) Dry at 60° C. for 20 minutes.
(11) Laminate the coated release liner layer with 3M Scotchpak 9744, 3.0 mil release liner.
(12) Apply 4-pound roller 2 times for better contact.
(13) Remove release liner from the Naltrexone laminate as well as one of the release liner from the Barrier Layer laminate.
(14) Laminate both layers together i.e. Naltrexone laminate with Barrier Layer laminate.
(15) Apply 4-pound roller 2 times for better contact.
(16) Remove release liner from the Oxymorphone laminate.
(17) Remove release liner from the combined layers of Naltrexone laminate with Barrier Layer laminate.
(18) Laminate both layers together i.e. Oxymorphone laminate with combined layers of Naltrexone laminate with Barrier Layer laminate.
(19) Apply 4-pound roller 2 times for better contact.
(20) Die cut patches of appropriate sizes for the assay, patch weight, patch thickness and for skin permeation.

### Patches Comprising 3% Oxymorphone

### Flux Values

Next the flux values of both Oxymorphone and Naltrexone through male white skin (69 years, skin bank MM041115) were determined for a transdermal patch comprising:
a first layer comprising 3% oxymorphone, 5% linoleic acid and a Durotak-87-4287 adhesive;
a barrier layer of 80% ethyl cellulose and 20% hydroxypropyl cellulose; and
a second layer comprising 3% Naltrexone and a Durotak-87-2054 adhesive.

The results in Table 8 show that the above transdermal patch displayed an excellent level of Oxymorphone flux whilst maintaining no flux of Naltrexone.

TABLE 8
Flux values of Oxymorphone and Naltrexone for multilaminate,
oxymorphone and naltrexone transdermal patches of the present invention.

| | | OXY FLUX or naltrexone flux |
|---|---|---|
| | | determined using male white skin |
| | Coating | age 69 Section PT |
| Formulation | gap | (μg/cm²/h) |
| 3% Naltrexone in | 0.400 mm | Average flux 24-72 hrs = 6.37 |
| Durotak 87-2054 | | (OXY) |
| Barrier layer (80% Ethyl | 0.400 mm | Average flux 0-72 hrs = 5.67 |
| cellulose + 20% HPC | | (OXY) |
| 3% oxymorphone in | 0.600 mm | NO NALTREXONE |
| Durotak 87-4287 and 5% | | PRERMEATED |
| linoleic acid | | |
| 1% Naltrexone in | 0.400 mm | NO NALTREXONE |
| Durotak 87-2054 | | PRERMEATED |
| Barrier layer (80% Ethyl) | 0.400 mm | |
| cellulose + 20% HPC | | |
| Placebo layer (Durotak- | 0.400 mm | |
| 87-2054) | | |
| 3% Naltrexone in | 0.400 mm | NO NALTREXONE |
| Durotak 87-2054 | | PRERMEATED |
| Barrier layer (80% Ethyl | 0.400 mm | |
| cellulose + 20% HPC | | |
| Placebo layer (Durotak- | 0.400 mm | |
| 87-2054) | | |
| 3% Naltrexone in | 0.400 mm | NO NALTREXONE |
| Durotak 87-2054 | | PRERMEATED |
| Barrier layer (80% Ethyl | 0.400 mm | |
| cellulose + 20% HPC | | |
| Placebo layer (Durotak- | 0.400 mm | |
| 87-4287 | | |
| 3% oxymorphone in | 0.600 mm | Average flux 24-72 hrs = 6.95 |
| Durotak 87-4287 and 5% | | (OXY) |
| linoleic acid | | Average flux 0-72 hrs = 6.13 |
| | | (OXY) |
| 3% Naltrexone in | 0.400 mm | Average flux 0-72 hrs = 0.602 |
| Durotak 87-2054 | | (NTX) |

### Patches Comprising 2% and 2.5% Oxymorphone

### Flux Values

Using patches comprising 2% and 2.5% Oxymorphone, the flux values of Oxymorphone through male white skin (67 years, skin bank JA040115) were determined. The results are summarised in Table 9 below.

TABLE 9
Flux values of Oxymorphone for transdermal patches comprising
2% and 2.5% Oxymorphone.

| | | | FLUX VALUE | |
|---|---|---|---|---|
| | Amount of | COATING | (t 24-72 hrs; | |
| | Oxymorphone | GAP | units: | |
| % OXYMORPHONE | per cm² | (mm) | μg/cm²/h) | COMMENT |
| 2.5% oxymorphone | 540 μg/cm² | 0.5 mm + | 7.56 (a) | SKIN JA040115 |
| in Durotak 87-4287 | | 0.5 mm | 7.64 (b) | Age 67 LPL |
| and 5% linoleic acid | | Total = | 7.43 (c) | |
| | | 1.0 mm | Average of 3 | |
| | | | 7.54 μg/cm²/hr | |
| 2% oxymorphone in | 434 μg/cm² | 0.5 mm + | 4.31(a) | SKIN JA040115 |
| Durotak 87-4287 and | | 0.5 mm | 5.22 (b) | Age 67 LPL |
| 5% linoleic acid | | Total = | 5.35 (c) | |
| | | 1.0 mm | Average of 3 | |
| | | | 4.96 μg/cm²/hr | |

### Transdermal Patches Prepared by Double Coating Process

Transdermal patches containing greater amounts of Oxymorphone and Naltrexone (greater thickness of appropriate layers) were next prepared and the flux values of Oxymorphone and Naltrexone through male white skin (69 years, skin bank MM041115) were determined. The patches were made using similar general procedures to the procedure described in paragraph [00140] above, albeit two coatings of each layer (e.g. Oxymorphone layer, Naltrexone layer and barrier layer) were employed.

The flux values obtained for these ‘thicker’ patches are summarised in Tables 10 to 15 below.

Tables 10 and 11—Flux values of Qxymorphone tor a transdermal patches comprising 2.5% Qxymorphone.

TABLE 10

| | | | FLUX VALUE | |
|---|---|---|---|---|
| | Amount | COATING | (t 24-72 hrs; | |
| | Oxymorphone | GAP | units: | |
| % OXYMORPHONE | per cm² | (mm) | μg/cm²/h) | COMMENT |
| 2.5% oxymorphone | 635 μg/cm² | 0.6 mm + | 4.93 (a) | SKIN |
| in Durotak 87-4287 | | 0.6 mm | 7.08 (b) | MM041115 |
| and 5% linoleic acid | | Total = | 6.83(c) | Age 69 LPL |
| | | 1.2 mm | 7.46 (d) | |
| | | | 7.18 (e) | |
| | | | Average of 5 | |
| | | | 6.69 μg/cm2/hr | |

TABLE 11

| | | | FLUX | |
|---|---|---|---|---|
| | | | VALUE | |
| | Amount | COATING | (t 24-72 hrs; | |
| | Oxymorphone | GAP | units: | |
| % OXYMORPHONE | per cm² | (mm) | μg/cm²/h) | COMMENT |
| 2.5% oxymorphone | 587 μg/cm² | 1.2 mm | 5.06 (a) | SKIN |
| in Durotak 87-4287 | | (10.6 mil) | 5.77 (b) | JL080915 |
| and 5% linoleic | | | 2.58 (c) | Age 52 |
| acid | | | Average of 3 | RPL |
| | | | 4.47 μg/cm2/hr | |

Tables 12 and 13—Flux values of Naltrexone for transdermal patches comprising 3.0% Naltrexone.

TABLE 12

| | Amount | | | |
|---|---|---|---|---|
| | Naltrexone per | COATING | FLUX VALUE | |
| % NALTREXONE | cm² | GAP | (t 24-72 hrs) | COMMENT |
| 3% in Durotak 87-2054 | 848 μg/cm² | 0.5 mm + | 0.20 (a) | SKIN |
| | | 0.5 mm | 0.22 (b) | MM041115 |
| | | Total = | 0.34(c) | Age 69 LPL |
| | | 1.0 mm | 0.3 (d) | |
| | | | 0.32 (e) | |
| | | | Average of 5 | |
| | | | 0.28 μg/cm2/hr | |

TABLE 13

| | Amount | | | |
|---|---|---|---|---|
| | Naltrexone per | COATING | FLUX VALUE | |
| % NALTREXONE | cm² | GAP | (t 24-72 hrs) | COMMENT |
| 3% in Durotak 87-2054 | 613 μg/cm² | 0.8 mm | 0.80 (a) | SKIN |
| | | | 1.07 (b) | JL080915 |
| | | | 1.65(c) | Age 52 |
| | | | Average of 3 | RPL |
| | | | 1.17 μg/cm2/hr | |

Tables 14 and 15—Flux values of Oxymorphone and Naltrexone for multilaminate transdermal patches of the present invention

TABLE 14

| | Amount | | | |
|---|---|---|---|---|
| | Oxymorphone/ | COATING | FLUX | |
| TRILAYERED | Naltrexone per | GAP | VALUE | |
| FORMULATION | cm² | (mm) | (t 24-72 hrs) | COMMENT |
| 3% Naltrexone in | NAL | 0.5 + 0.5 mm | NTX | SKIN |
| Durotak 87-2054 | 9.12 mg/10.75 cm² | | No | MM041115 |
| Barrier layer | (33.93/40 mg²) | 0.4 mm | Naltrexone | Age 69 LPL |
| comprising 80% | 848 μg/cm² | | detected | |
| EC and 20% HPC | | | | |
| 2.5% | OXY | 0.6 + 0.6 mm | OXY | |
| Oxymorphone in | 6.83 mg/10.75 cm² | | 6.23 (a) | |
| Durotak 87-4287 | (25.41/40 mg²) | | 5.40 (b) | |
| and 5% linoleic | 635 μg/cm² | | 5.89(c) | |
| acid | | | 6.00 (d) | |
| Backing layer SP | | | 5.99 (e) | |
| 9733 | | | Average of 5 | |
| Release liner SP | | | 5.9 μg/cm²/hr | |
| 9744 | | | | |

TABLE 15

| | Amount | | | |
|---|---|---|---|---|
| | Oxymorphone/ | COATING | FLUX | |
| TRILAYERED | Naltrexone per | GAP | VALUE | |
| FORMULATION | cm² | (mm) | (t 24-72 hrs) | COMMENT |
| 3% Naltrexone | NAL | 0.4 + 0.4 mm | NTX | SKIN |
| in Durotak 87- | 6.59 mg/10.75 cm² | | No | JL080915 |
| 2052 | (24.52/40 mg²) | | Naltrexone | Age 52 |
| Barrier layer | 613 μg/cm² | 0.4 mm | detected | RPL |
| comprising 80% | | | | |
| EC and 20% | | | | |
| HPC | | | | |
| 2.5% | OXY | 0.6 + 0.6 mm | OXY | |
| Oxymorphone in | 6.31 mg/10.75 cm² | | 5.81 (a) | |
| Durotak 87- | (23.48/40 mg²) | | 6.32 (b) | |
| 4287 and 5% | 587 μg/cm² | | 4.89 (c) | |
| linoleic acid | | | Average of 3 | |
| Backing layer | | | 5.67 μg/cm²/hr | |
| SP 9733 | | | | |
| Release liner | | | | |
| SP 9744 | | | | |

### Scale-Up Production

A bulk synthesis of transdermal patches were next prepared.

A total of 4026×10 cm² transdermal patches were prepared, with each containing: 1.72 mg of oxymorphone with a dry weight coating thickness of approximately 2.7 mil (using a coating gap of 0.340 mm); a barrier layer with a dry weight thickness of approximately 1.3 mil; and 1.06 mg of naltrexone with a dry weight thickness of 1.4 mil.

The flux values for the ‘scale-up’ batch of transdermal patches are provided in Tables 16 and 17 below.

TABLE 16
Flux values of Oxymorphone for a scale-up monolayer transdermal patch comprising
2.5% Oxymorphone

| | | | FLUX | |
|---|---|---|---|---|
| | | | VALUE | |
| | Amount | COATING | (t 24-72 hrs; | |
| | Oxymorphone | GAP | units: | |
| % OXYMORPHONE | per cm² | (mm) | μg/cm²/h) | COMMENT |
| 2.5% oxymorphone | 173 μg/cm² | 0.340 mm | 2.91 (a) | SKIN |
| in Durotak 87-4287 | | | 3.17 (b) | CM120115 |
| and 5% linoleic acid | | | 2.98 (c) | Age 48 |
| | | | 2.92 (d) | White Male |
| | | | 2.95 (e) | LPL |
| | | | Average of 5 | |
| | | | 2.99 μg/cm²/hr | |

TABLES 17
Flux values of Oxymorphone and Naltrexone for the scale-up multilaminate
transdermal patches of the present invention

| | Amount | | | |
|---|---|---|---|---|
| | Oxymorphone/ | COATING | FLUX | |
| TRILAYERED | Naltrexone per | GAP | VALUE | |
| FORMULATION | cm² | (mm) | (t 24-72 hrs) | COMMENT |
| 3% Naltrexone in | NAL | 0.110 mm | NTX | SKIN |
| Durotak 87-2052 | 1.06 mg/10.00 cm² | (1.4 mil) | No | CM120115 |
| | | | Naltrexone | Age 48 |
| | | | detected | White male |
| | | | | LPL |
| Barrier layer | | 0.380 mm | | |
| comprising 80% | | (1.3 mil) | | |
| EC and 20% HPC | | | | |
| 2.5% | OXY | 0.34 mm | OXY | |
| Oxymorphone in | 1.73 mg/10.00 cm² | (2.7 mil) | 2.46 (a) | |
| Durotak 87-4287 | 173 μg/cm² | | 2.80 (b) | |
| and 5% linoleic | | | 2.64 (c) | |
| acid | | | 2.60 (d) | |
| Backing layer SP | | | 2.44 (e) | |
| 9733 | | | 2.79 (f) | |
| Release liner SP | | | Average of 6 | |
| 9744 | | | 2.62 μg/cm²/hr | |

While specific embodiments of the invention have been described for the purpose of reference and illustration, various modifications will be apparent to a person skilled in the art without departing from the scope of the invention as defined by the appended claims.

What is claimed is:

1. A transdermal patch comprising a multilaminate, wherein said multilaminate comprises:
(i) a first layer, comprising:
an opioid analgesic, or a pharmaceutically acceptable salt thereof;
a pharmaceutically acceptable pressure sensitive adhesive; and
optionally a penetration enhancer;
(ii) a second layer, comprising:
an opioid antagonist, or a pharmaceutically acceptable salt thereof; and
a pharmaceutically acceptable adhesive;
(iii) a barrier layer, comprising:
a water soluble cellulose derivative and a water insoluble cellulose derivative;
wherein said barrier layer is disposed between the first and second layers, and wherein the barrier layer substantially prevents the opioid antagonist diffusing from the second layer to the first layer during use;
with the proviso that the barrier layer is devoid of polyethylene glycol.

2. A transdermal patch according to claim 1, wherein the opioid analgesic is selected from the group consisting of buprenorphine, fentanyl, hydromorphone, oxycodone, tapentadol, and oxymorphone, or a pharmaceutically acceptable salt thereof.

3. (canceled)

4. A transdermal patch according to claim 1, wherein the opioid analgesic is present at an amount of 1-15% w/w or at an amount of 2-4% w/w in the first layer.

5. (canceled)

6. A transdermal patch according to claim 1, wherein the total amount of adhesive will constitute between 58 and 99% w/w or between 85 and 99% w/w of the first and second layers respectively.

7. (canceled)

8. A transdermal patch according to claim 1, wherein the pressure sensitive adhesive of the first layer is a pressure sensitive polyacrylate adhesive.

9. A transdermal patch according to claim 8, wherein the pressure sensitive polyacrylate adhesive of the first layer has a viscosity of between 1600 and 19000 mPa.

10. A transdermal patch according to claim 8, wherein the pressure sensitive polyacrylate adhesive of the first layer comprises no functional groups or a plurality of hydroxyl functional groups.

11. A transdermal patch according to claim 8, wherein the pressure sensitive polyacrylate adhesive of the first layer is Durotak-87-4287.

12. A transdermal patch according to claim 1, wherein the pharmaceutically acceptable adhesive of the second layer is a pharmaceutically acceptable polyacrylate adhesive.

13. A transdermal patch according to claim 12, wherein the pharmaceutically acceptable polyacrylate adhesive of the second layer has a viscosity of between 1600 and 10000 mPa.

14. A transdermal patch according to claim 12, wherein the pharmaceutically acceptable polyacrylate adhesive of the second layer is Durotak-87-2054.

15. A transdermal patch according to claim 1, wherein the first layer comprises a penetration enhancer.

16. A transdermal patch according to claim 15, wherein the penetration enhancer of the first layer is oleic or linoleic acid.

17. (canceled)

18. A transdermal patch according to claim 15, wherein the penetration enhancer is present in an amount of 2-12% w/w or in an amount of 4-7% w/w of the first layer.

19. (canceled)

20. A transdermal patch according to claim 1, wherein the opioid antagonist is present in an amount of 0.1-5% w/w or in an amount of 1-3% w/w of the second layer.

21. (canceled)

22. A transdermal patch according to claim 1, wherein the opioid antagonist in the second layer is naltrexone (“NTX”), naloxone (“NLX”), nalmefene or a pharmaceutically acceptable salts thereof.

23. (canceled)

24. A transdermal patch according to claim 1, wherein the barrier layer is devoid of any additional hydrophilic polymer (e.g. polyethylene glycol, polyvinyl alcohol or polyacrylic acid).

25. A transdermal patch according to claim 1, wherein the barrier layer consists essentially of a water soluble cellulose derivative and a water insoluble cellulose derivative.

26. A transdermal patch according to claim 1, wherein greater than 95% w/w of the barrier layer is comprised of a water soluble cellulose derivative and a water insoluble cellulose derivative.

27. A transdermal patch according to claim 1, wherein the barrier layer comprises between 10-30% hydroxypropylcellulose and between 70-90% ethylcellulose by weight on dry basis, or comprises 20% hydroxypropylcellulose and 80% ethylcellulose by weight on dry basis.

28. (canceled)

29. A transdermal patch according to claim 1, wherein the ratio of opioid analgesic:opioid antagonist is between 10:1 and 1:10 or between 4:1 and 1:4.

30.-32. (canceled)

33. A method of treating a condition selected form the group consisting of opioid dependence, alcohol dependence, polydrug addiction, pain, cocaine addiction, eating disorders (e.g., binge eating) and treatment-resistant depression in a subject in need of such treatment, said method comprising administering a transdermal patch according to claim 1 to the skin of the subject.
